(19) United States
(12) Patent Application Publication
(10) Pub. No.: US 2026/0232221 A1
(43) Pub. Date: Aug. 13, 2026

(54) MONITORING SYSTEM USING PERSONALIZED PHYSICS ENGINE

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/536,952

(22) Filed: Feb. 11, 2026

Related U.S. Application Data
(60) Provisional application No. 63/757,086, filed on Feb. 11, 2025.

(51) Int. Cl. A61B 5/11; A61B 5/00; A61B 5/0205

(52) U.S. Cl. CPC A61B 5/11; A61B 5/0205; A61B 5/1112; A61B 5/7264

(57) ABSTRACT

Systems, methods and apparatuses are provided for implementing personalized physics engines. An example system includes a sensor-enabled environment containing a person under monitoring, a machine learning system, and a digital twin of the person under monitoring, wherein the machine learning system is trained to update the digital twin to represent a baseline behavior of the person under monitoring based upon data from the sensor-enabled environment about behaviors of the person under monitoring, and the machine learning system is configured to trigger an alert responsive to a behavior of the person under monitoring departing from a corresponding behavior of the digital twin by a predetermined amount.

### CROSS REFERENCE TO RELATED APPLICATION

This application claims priority to U.S. Provisional Patent Application No. 63/757,086 filed on Feb. 11, 2025, the disclosure of which is incorporated by reference herein in its entirety.

### BACKGROUND

Persons under care are often monitored together with their environments, and other persons interacting with them. Monitoring may be used to determine patient condition, predict and reduce risk, improve provision of care and quality of life, and other purposes. Many different types of sensors, including cameras, worn sensors, other environmental sensors, and the like are employed in different applications. These sensors produce an enormous amount of data that can be difficult to exploit in a practical manner.

### SUMMARY

Certain aspects of some embodiments disclosed herein are set forth below. These aspects are presented merely to provide the reader with a summary of certain forms the invention might take and that these aspects are not intended to limit the scope of the invention. Indeed, the invention may encompass a variety of aspects that may not be set forth below.

Embodiments of the present disclosure generally relate to a system that may include a plurality of sensors monitoring a person under monitoring (PUM) and an environment in which the PUM is located; a machine learning (ML) monitoring system to receive data from the plurality of sensors and identify, using the data received from the plurality of sensors, a movement type and a set of values of movement parameters corresponding to a movement of the PUM, and to produce a movement representation representing the movement type and the set of values of movement parameters corresponding to the movement; and a ML analysis system configured to receive the movement representations from the ML monitoring system, based at least in part on the movement representations, to identify and report changes in a condition of the PUM.

In some embodiments, the system may optionally include a data repository storing historical movement data for the PUM, the ML analysis system in communication with the memory, the ML analysis system is further configured to identify changes in the condition of the PUM based on a comparison of the movement representations with the historical movement data for the PUM.

In some embodiments, the system may optionally include a data repository storing movement representations for a plurality of individuals, the ML analysis system is further configured to identify changes in the condition of the PUM based on correlating the movement of the PUM to the movement representations for the plurality of individuals.

In some embodiments, the system may optionally include that the movement type is selected from the group consisting of at least one of: (a) movement of a particular joint, (b) walking, (c) standing from a seated position, (D) sitting down from a standing position, (e) getting up from floor, (f) getting out of bed, (g) sitting up in bed, (h) reaching for an object, (i) picking up an object, (j) picking up an object from the floor, (k) gait changes, (l) limb tremors, (m) posture transitions, and (n) contacting an object.

In some embodiments, the system may optionally include that the movement parameters include one or more parameters selected from the group consisting of movement duration, movement distance, movement starting location, movement ending location, PUM body position prior to movement, PUM body position after movement, movement speed, movement vector, movement force, joint angles, angular velocity, acceleration, force vector, and energy expenditure.

In some embodiments, the system may optionally include that the movements representations include information regarding a joint-angle range or max achievable angle for a particular joint of the PUM.

In some embodiments, the system may optionally include a non-transitory memory storing a digital twin including a software model of the human musclo-skeletal system, the ML monitoring system is trained using the software model and configured to apply the movements representations to produce a musculoskeletal software model customized to the PUM.

In some embodiments, the system may optionally include a controller configured to reconfigure one or more of the plurality of sensors based on the identified movement type, reconfiguring one or more of the plurality of sensors includes an action selected from the group consisting of activating a sensor, deactivating a sensor, changing a sampling rate of a sensor, changing a granularity of data collected by a sensor, and changing a focus of a sensor.

In some embodiments, the system may optionally include that the movement representation includes one or more machine-interpretable tokens or vectors encoding the identified movement type and the set of values of movement parameters.

In some embodiments, the system may optionally include that the ML analysis system is further configured to compute a deviation metric between the movement representations and a baseline movement profile of the PUM and trigger an alert when the deviation metric exceeds an adaptive threshold.

In some embodiments, the system may optionally include that the ML analysis system is further configured to output a risk metric based on the condition of the PUM, the risk metric comprises a signal including at least one of a potential fall of the PUM, a deterioration in mobility in the PUM, an increased risk condition in the PUM, a change from a baseline behavior of the in the PUM, or an emergency that warrants human and/or automated response.

In some embodiments, the system may optionally include that the sensors are selected from the group consisting at least one selected from the group consisting of: motion sensors, mmWave radars, cameras, WiFi signal-based location sensors, wearable accelerometers, gyroscopes, altimeters, microphones, pressure mats, radar-Doppler sensors, capacitive sensors, 3D scanners, inertial measurement units (IMUs), haptic sensors, and biometric sensors.

In another aspect, embodiments of the present disclosure generally relate to a method that may include receiving data from a plurality of sensors monitoring a person under monitoring (PUM) and an environment in which the PUM is located; determining from the data a movement type and a set of values of movement parameters corresponding to a movement of the PUM; storing a movement representation representing the movement type and the set of values of movement parameters corresponding to the movement; determining from the movement representations changes in a condition of the PUM.

In some embodiments, the method may optionally include that the movement type and the set of movement-parameter values are determined using a movement analysis machine learning system.

In some embodiments, the method may optionally include training the movement analysis machine learning system using a Musculo-skeletal computational model of a human body.

In some embodiments, the method may optionally include producing a customized musculoskeletal computational model to the PUM based applying the movements representations to the Musculo-skeletal computational model.

In some embodiments, the method may optionally include that changes in the condition of the PUM are determined using a machine learning (ML) analysis system by comparing the tokenized movement representations to a threshold derived from a quiescent baseline.

In some embodiments, the method may optionally include receiving the data from the plurality of sensors for a time period; and determining a segment of the time period which corresponds to the movement of the PUM prior to determining the movement type and the set of movement-parameter values.

In some embodiments, the method may optionally include receiving the data from the plurality of sensors for a time period; and determining a segment of the time period which corresponds to a predetermined type of movement.

In some embodiments, the method may optionally include reconfiguring one or more of the plurality of sensors based on the identified movement type by at least one action selected from the group consisting of activating a sensor, deactivating a sensor, changing a sampling rate of a sensor, changing focus of a sensor, and changing a granularity of data collected by a sensor.

In some embodiments, the method may optionally include outputting a risk metric based on the condition of the PUM, the risk metric comprises a signal selected from the group consisting of a potential fall of the PUM, a deterioration in mobility in the PUM, an increased risk condition in the PUM, a change from a baseline behavior of the in the PUM, and an emergency that warrants human and/or automated response.

In some embodiments, the method may optionally include computing a deviation metric between the movement representations and a baseline movement profile of the PUM; and triggering an alert when the deviation metric exceeds a predetermined threshold.

In some embodiments, the method may optionally include encoding the identified movement type and the set of values of movement parameters in one or more machine-interpretable tokens or vectors as part of the movement representation.

In some embodiments, the method may optionally include that the sensors are selected from the group consisting at least one of: motion sensors, mm Wave radars, cameras, WiFi signal-based location sensors, wearable accelerometers, gyroscopes, altimeters, microphones, pressure mats, radar-Doppler sensors, capacitive sensors, 3D scanners, inertial measurement units (IMUs), haptic sensors, and biometric.

In yet another aspect, embodiments of the present disclosure generally relate to a system that may include a sensor-enabled environment containing a person under monitoring (PUM); a machine learning (ML) analysis system; and a digital twin of the PUM, the ML analysis system trained to update the digital twin to represent a baseline behavior of the PUM based upon data from the sensor-enabled environment about behaviors of the PUM, and the ML analysis system is configured to trigger an alert responsive to a behavior of the PUM departing from a corresponding behavior of the digital twin by a predetermined amount.

In some embodiments, the system may optionally include a machine learning (ML) data handling system configured to accept data from the sensor-enabled environment as input and to output the data as one or more keys, vectors, or input tokens for the ML analysis system.

In some embodiments, the system may optionally include a machine learning (ML) data handling system configured to accept data from the sensor-enabled environment as input and to categorize one or more segments of the data as a movement artifact.

In some embodiments, the system may optionally include that the ML data handling system is configured to remove a data segment corresponding to a movement artifact before sending the data to the ML analysis system.

In some embodiments, the system may optionally include that the ML analysis system is configured to analyze the movement artifact from the ML data handling system and determine a physics-based validity or invalidity of the movement artifact.

In some embodiments, the system may optionally include a machine learning (ML) system configured to accept output data from the ML analysis system, the ML system functions as an augmentation stage of a retrieval augmented generation (RAG) process, and the ML system is configured to categorize outputs of the ML analysis system as a generation stage of the RAG process.

In other aspects, embodiments of the present disclosure generally relate to a computer-implemented method of monitoring a person under monitoring (PUM), the method may include receiving, from sensors of a sensor-enabled environment (SEE), data indicative of behaviors of the PUM; updating, by a machine learning (ML) system, a digital twin of the PUM to represent a baseline behavior of the PUM based upon the data from the SEE; and triggering, by the ML system, an alert responsive to a behavior of the PUM departing from a corresponding behavior of the digital twin by at least a predetermined amount.

In some embodiments, the method may optionally include accepting, by a second ML system, at least a portion of the data from the SEE as input; and outputting, by the ML system, one or more keys, vectors, or input tokens used as inputs to the second ML system that updates the digital twin.

In some embodiments, the method may optionally include accepting, by a second ML system, at least a portion of the data from the SEE as input; and categorizing, by the second ML system, one or more segments of the data as a movement artifact.

In some embodiments, the method may optionally include removing, by the second ML system, the one or more segments categorized as movement artifacts prior to providing the data to the ML system that updates the digital twin.

In some embodiments, the method may optionally include analyzing, by the second ML system, the movement artifact categorized by the ML system and determining a physics-based validity or invalidity of the movement artifact.

In some embodiments, the method may optionally include providing, from the ML system, output data as an augmentation stage of a retrieval-augmented generation (RAG) process; and categorizing, by the machine learning system, the output data as a generation stage of the RAG process.

In yet other aspects, embodiments of the present disclosure generally relate to a system that may include one or more processors; a sensor-enabled environment (SEE) containing a person under monitoring (PUM), the SEE including a plurality of sensors configured to generate multimodal measurements of movements of the PUM; one or more first-stage models configured to transform at least a portion of the multimodal measurements into tokenized movement representations; a personalized physics engine (PPE) implemented by the one or more processors and configured to apply physics-based constraints personalized to the PUM including at least joint-angle bounds and contact-surface interactions, identify and filter movement artifacts in the tokenized movement representations, and output artifact-reduced movement data; a digital twin of the PUM implemented by the one or more processors and configured to update a baseline behavior model of the PUM using the artifact-reduced movement data; and an alerting module configured to compute a deviation metric between a current behavior of the PUM and the baseline behavior model and to trigger an alert responsive to the deviation metric exceeding a predetermined threshold.

In some embodiments, the system may optionally include that the sensors are selected from the group consisting of: motion sensors, mm Wave radars, cameras, WiFi signal-based location sensors, wearable accelerometers, gyroscopes, and altimeters.

In some embodiments, the system may optionally include that measurements from the plurality of sensors are fused using a Kalman filter and a particle filter to correct for sensor noise and inaccuracies.

In some embodiments, the system may optionally include that the first-stage model includes a transformer configured to output embeddings for fixed-length windows of the multimodal measurements.

In some embodiments, the system may optionally include that the joint-angle bounds include a knee-flexion bound and a neck-rotation bound of the PUM, and the PPE is configured to reject token sequences that violate the bounds.

In some embodiments, the system may optionally include that the contact-surface interactions include at least one of a compliant surface selected from (a) a piece of furniture, (b) a cooking appliance, (c) a floor, (d) a wall, (e) door, and (f) a mobile electronic device.

In some embodiments, the system may optionally include identifying the movement artifacts include evaluating frequency-domain features including tremor frequencies in a range of 4 Hz to 12 Hz and down-weighting corresponding windows before updating the baseline behavior model.

In some embodiments, the system may optionally include that the digital twin represents reachable limb positions as a manifold in a higher-dimensional space and constrains predictions.

In some embodiments, the system may optionally include that the alerting module is configured to compute a deviation metric in an embedding space between the current behavior and the baseline behavior model.

In some embodiments, the system may optionally include that the alerting module is configured to compute the adaptive threshold using an exponential moving average over quiescent intervals within a trailing multi-day window.

In some embodiments, the system may optionally include that the recommended mitigation includes at least one of: (a) providing a haptic, audio, or visual instruction to the PUM, (b) adjusting a configuration of at least one sensor in the SEE, and (c) notifying a caregiver.

In some embodiments, the system may optionally include a repository storing labeled descriptors associating movements with textual intent descriptors, and wherein the first-stage model is configured to map tokenized movement representations to the textual intent descriptors.

In some embodiments, the system may optionally include that the first-stage model and the PPE are configured to operate as an augmentation stage of a retrieval-augmented generation pipeline.

In some embodiments, the system may optionally include a first machine learning (ML) system configured to generate a generation stage of the pipeline; a second machine learning (ML) system receiving outputs from the generation stage; and a third machine learning (ML) system consuming outputs of the second ML system.

In some embodiments, the system may optionally include that the SEE is configured to identify furniture and obstacles, and the digital twin assigns risk scores to predicted contact areas based on the furniture and obstacles.

In some embodiments, the system may optionally include that a communication between the sensors, the first-stage model, the PPE, and the digital twin is encapsulated in authenticated secure tokens permitting access by authorized modules.

In some embodiments, the system may optionally include that the tokenized movement representations include vectors including contextual tags indicating at least time-of-day and room identifiers.

In some embodiments, the system may optionally include that the first-stage model includes a graph neural network encoding joint connectivity and a range of limb movement.

In some embodiments, the system may optionally include the first-stage model includes a recurrent neural network configured to predict subsequent movements from prior movements in a sequence.

In some embodiments, the system may optionally include that the digital twin includes a physics simulation of the PUM.

In yet another aspect, embodiments of the present disclosure generally relate to a method that may include receiving, from sensors of a sensor-enabled environment (SEE), multimodal measurements characterizing movements of a person under monitoring (PUM); generating, by a first-stage model, tokenized movement representations from the multimodal measurements; filtering, by a personalized physics engine (PPE), movement artifacts and movements failing physics-based constraints of the PUM to produce artifact-reduced movement data; updating, by a digital twin of the PUM, a baseline behavior model using the artifact-reduced movement data; determining, by comparison of a current behavior to the baseline behavior model, a deviation metric; and triggering an alert when the deviation metric exceeds predetermined threshold.

In some embodiments, the method may optionally include synchronizing measurements from a plurality of sensors using time-stamps and resampling to a common window prior to generating the tokenized movement representations.

In some embodiments, the method may optionally include producing the artifact-reduced movement data by at least one action selected from the group consisting of (a) applying joint-angle constraints, (b) analyzing contact-surface models, and (d) performing an artifact detection procedure.

In some embodiments, the method may optionally include that updating the baseline behavior model includes applying an exponential moving average over artifact-free windows.

In some embodiments, the method may optionally include transmitting, with the alert, a mitigation including at least action selected from the group consisting of: (a) instructing the PUM to pause activity, (b) requesting assistance from a caregiver, and (c) increasing sampling rate of one or more sensors.

In some embodiments, the method may optionally include adjusting the predetermined threshold based on at least one factor selected from the group consisting of (a) energy expenditure of the PUM, (b) measured fatigue of the PUM, (c) historical circadian behavior of the PUM, and (d) movement success metrics of the PUM.

In other aspects, embodiments of the present disclosure generally relate non-transitory machine-readable medium storing instructions that, when executed by one or more processors, cause the one or more processors to perform operations including any of the methods.

Various refinements of the features noted above may exist in relation to various aspects of the present embodiments. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the summary presented above is intended only to familiarize the reader with certain aspects and contexts of some embodiments without limitation to the claimed subject matter.

### BRIEF DESCRIPTION OF THE FIGURES

Various example embodiments are provided in the following Figures and the detailed description thereof which follows below. It will be appreciated that these Figures are provided for illustrative purposes only, and that embodiments of the present disclosure are in no way intended to be limited by inclusions or exclusions of particular features within the various Figures herein.

FIG. 1 illustrates an example system employing a motion model, according to example embodiments of the present disclosure.

FIG. 2 illustrates an example system including a first stage ML model, a personalized physics engine, and an additional ML model in a multi-stage processing architecture, according to example embodiments of the present disclosure.

FIG. 3 illustrates an example system including a configured personalized physics engine, according to example embodiments of the present disclosure.

FIG. 4 illustrates an example system including personalized monitoring, according to example embodiments of the present disclosure.

FIG. 5 illustrates an example system including digital twins, according to example embodiments of the present disclosure.

FIG. 6 illustrates an example system including ML monitoring and analysis systems, according to example embodiments of the present disclosure.

FIG. 7 illustrates a flowchart of an example method for monitoring a person in a sensor-enabled environment, according to example embodiments of the present disclosure.

FIG. 8 illustrates a flowchart of an example method for monitoring a person in a sensor-enabled environment, according to example embodiments of the present disclosure.

FIG. 9 illustrates example data structures including a movement module, according to example embodiments of the present disclosure.

FIG. 10 illustrates example data structures including a movement framework, according to example embodiments of the present disclosure.

FIG. 11 illustrates a block diagram of a computing system, according to example embodiments of the present disclosure.

### DETAILED DESCRIPTION

Various sensors, devices and/or systems may monitor a sensor-enabled environment (SEE). These sensors, devices and/or systems may provide data about objects in the environment to one or more computing systems for analysis. A physics engine may be a computerized model of physical behavioral aspects of objects. Physics engines may model various forces upon an object and/or subject, both external and internal, and model a behavior of that object in response to various conditions and force applications.

This disclosure generally describes the use of physics engines, which are based on the rules and laws of physics of the real-world environment that can be calibrated, customized, and configured to represent, at least in part, a person under monitoring (PUM) in a sensor-enabled environment (SEE). The engines may also represent, in a similar fashion, other people and/or objects in the SEE.

In some embodiments, a personalized physics engine (PPE) can be used to establish one or more baselines, represented for example, by a set of movements, that one or more stakeholders, including a PUM, may undertake in a SEE. This initial set of movements can be in the form of movement modules, for example represented as tokens and movement frameworks, which can represent the possible sequences and/or connections of the movement modules. For example, the movements modules may comprise multiple elements, for example movement modules for the joints in a limb, such as ankle, knee, hip movement modules for a leg, or wrist, elbow, shoulder movement modules for an arm.

In some embodiments, a SEE can comprise one or more sensors, devices and/or systems that are embedded in an environment, for example a room, house or other primary domicile of a PUM. The SEE can include one or more worn, carried and/or embedded sensors, devices and/or systems. The SEE can also include one or more systems for processing and/or managing the data sets generated by the one or more sensors, devices and/or systems present in an environment.

In some embodiments, a SEE can be considered as a single entity with multiple sensing capabilities, similar to a person tasked with monitoring another person. Within this approach there can be multiple sensing capabilities including, for example, visual, RADAR, LIDAR, audio, haptic, locational and/or temporal which can be used to identify the behavior of the person being monitored. In this example the SEE can replicate the monitoring of a PUM within the calibration, configuration and operations of the sensing capabilities of the one or more sensors, devices and/or systems present in the SEE, using for example one or more digital twin.

In some embodiments, data sets generated by the one or more sensors, devices and/or systems involved in the monitoring of an environment, for example a SEE, can form patterns, in whole or in part, that are representations of the activities, events and/or state of that environment, including any PUM and/or other stakeholders present therein.

These patterns can be representative of sets of repeated data that are generated by the one or more sensors, devices and/or systems present in a SEE. These repeated data sets can, in some embodiments, be organized into quantified sequences and/or elements, for example to represent a movement, activity, event or other state of the SEE and/or the stakeholders therein.

A movement module, in some embodiments can comprise the measured change in the location and/or orientation of at least one part of the human anatomy. Such change can include measurements of position of objects, such as body parts, in Euclidean space, force exerted by the person making the movement, variations in the orientation of the joints and/or limbs and may also include one or more estimation of the intention of such movement. For example, a movement module may correspond to a hand and wrist, where the movement of the hand, including the fingers and thumb, form an identifiable action, that is a movement. For example there can be movement modules that are identified and classified as, for example, grabbing, where the fingers and thumb curl around an object, for example a cup, holding, where the hand and palm support an object or can support an object passed to them, supporting, where the hand is oriented to achieve support for the person, such as with a hand rail, chair or other object.

A movement framework, in some embodiments, can comprise one or more movement modules, for example a movement framework for an arm may include movement modules for hand and wrist, forearm and elbow and upper and shoulder. A movement framework can comprise a sequence of movements, for example those involved in rising from sitting to standing. There can be multiple overlapping movement frameworks involving differing complexity of movement modules which can be used, in some embodiments, as part of the identification and monitoring of a PUM patterns and/or behaviors.

In some embodiments, a movement framework can also include one or more timing parameter, for example from the start of the movement framework to the conclusion of the movement framework can be, for example, a particular value in seconds, or a range of values between X and Y seconds.

Each movement framework can have one or more associated metrics representing the characteristics of the movement framework. For example there can be a success metrics, for example on a scale of 1 to 10, where if the PUM completes the movement framework within the time period allocated to that movement, then the metric may be high, whereas if the PUM exceeded the time period, or for example, attempted the movement and repeated parts of the movement, for example the sequence of movement modules is repeated, then the metric may be low. In some embodiments, these metrics may be in form of a matrix to represent multiple aspects of the movement modules and their formation of the movement framework.

In some embodiments, a human muscular skeletal representation can be used as a baseline for customized personalization of the PUM. For example, a scaled model of human muscular skeletal body can be used as a reference for a PUM, in that using one or more images of a PUM, the model can represent a baseline for their body. This can then be represented in a digital twin as the “reference” state of the PUM, in that none of the customization or personalization of the PUM has been added to that model. Although this gives a hypothetical state of the PUM, that is if all of their body was functioning as represented by the model, this state represents the full potential for their movements.

In some embodiments, a 3D scanner can be used to establish the skeletal muscular model for a PUM, where their body, in whole or in part, is scanned as part of the initialization process for the generation of their personalized physics engine (PPE). This can include the use of the reference model, which for example may represent an ideal skeletal muscular model for a PUM of that gender, age, height, weight and other physical characteristics and the 3D rendering of the actual PUM, such that the differences can be used as part of calculations of the movements of the PUM. For example, a hand-held 3D scanner may be used to create a measured scan of, for example, a limb of a PUM, the measurements of which, including scanning of a range of movements by that limb, can be integrated into the PPE.

In some embodiments where for example a PUM has undergone a procedure, for example a hip, knee or shoulder replacement or surgery, a 3D scan of the body part that has undergone the procedure and the initial movement of that part may be undertaken with a 3D scanner to generate an initial set of data from which their recuperation can be measured. In some situations, a 3D scanner can be employed in conjunction with the one or more sensors, devices and/or systems of a SEE to measure the range of motion of a PUM prior to any procedure. This data can then be used to train an AI/ML model, and/or configure one or more sensors, including for example a 3D scanner to measure the range and types of motions of the PUM after the procedure. For example, this can include where each of the human joints is represented as range of possible movements in three dimensions, the distance between the joints is anatomically correct and personalized for a specific PUM. This can include those limitations, such as the rotation of the neck being constrained to, for example to 180 degrees of movement that are part of the human physical condition. There can be other sets of constraints on movements of the PUM, where such movements have been measured by the one or more sensors, devices and/or systems of the SEE, including worn and/or carried sensors, such that the PUM PPE is configured, to match the current physical state of the PUM.

These movement modules and movement frameworks in any combination may be represented in one or more digital twins. The example system may employ various artificial intelligence or machine learning (AI/ML) Models such as large language models (LLMs), SLM (Small Language Models), LCM (large context models), LVM (large vison models), and/or specialized AI/ML models including other forms of deep learning and convolutional neural network models. For convenience, these may be referred to collectively, throughout the specification as “AI/ML Models,” unless a particular one of these types of models is called out with specificity. The digital twin together with one or more of the AI/ML models may be employed to determine, at least in part, a potential range of movements that these movement frameworks, which can comprise one or more movement modules and/or combinations thereof, can represent. In some embodiments, a physical muscular skeletal model can be calibrated and configured to the specifics of each of the movement modules and movement frameworks of a PUM as they are identified from the data sets generated by the one or more sensors, devices and/or systems comprising the SEE, including those worn and/or carried by the PUM.

Using a SEE to measure one or more data sets from the sensors, devices and/or systems present in the SEE can provide a set of measurements that include the movements of the PUM, represented for example as movement modules and/or movement frameworks, which can form the patterns and/or behaviors of the PUM, such that a PPE in a digital twin can replicate these movements of the PUM based at least in part on the measurements of the SEE.

This digital twin replication can, in some embodiments, be used to establish at least one control or baseline for the further measurements of the physical PUM in a SEE. For example, the baseline may represent the consistent and repeated movements of the PUM. In this manner a variance from a baseline to a current measured state of the movements and/or the behaviors they represent can be measured and identified. For example, this can include mapping the current measurements to one or more movement modules and/or movement frameworks, where the measured motions can, for example, match those of the previously measured, identified and stored movements. This matching can include one or more thresholds that are bound to, for example the previously identified movements, represented by movement modules, such that matching can include fuzzy logic and/or other variance evaluation based, at least in part, on these measurements and matching. This can include one or more patterns, where such patterns can form, in whole or in part the one or more behaviors of a PUM.

This approach addresses the difficulty of establishing the changes in the patterns and/or behaviors of a PUM over an extended period of time, for example daily, weekly, monthly, where for example, many indicators of potential wellness, health and/or safety of a PUM that may result in a detrimental event can be identified by these, often gradual changes. In many current circumstances these changes over extended periods are ignored by point or single data monitoring systems, often being represented as latent data or noise, rather than as the one or more informative signal that they can represent. The use of a SEE, combined with digital twins and a PPE addresses this shortcoming of current monitoring systems. For example, if a PUM has a hand that shakes and the degree of movement of the shake increases over time, this can indicate, for example, an increased risk of deterioration of a neurological disorder such as Parkinsons disease and/or may be an indication of lack of sleep, too much coffee or other possible causations. The use of AI/ML models, trained on the data sets provided by a SEE, in combination with PPE and digital twins can provide early indications of these potential events, which can include risk metrics for same, so that avoidance and/or mitigation strategies can be employed, which in some embodiments, can Include providing alerts and other communications to, for example the PUM, carer, medical professional and/or other stakeholders, including one or more systems for monitoring the PUM.

In some embodiments, in part dependent on the healthcare profile (HCP) of the PUM, one or more game theory engines can be employed to identify, determine and/or evaluate these indicators and/or the one or more strategies for avoidance and/or mitigation of potential impacts can be evaluated so as to determine the optimum strategy.

The use of a PPE and one or more digital twins to create a personalized representative replication of a PUM, where the movements of the PUM as represented by the PPE, can, in some embodiments, be used to mimic one or more sets of movements that represent the condition of the PUM and/or the observable measured behaviors thereof.

The combination, in whole or in part, of a SEE, PPE, digital twin, AI/ML models, one or more of which may be configured as a personalized representation of one or more stakeholder and/or one or more game theory engines and the games thereof, can represent the control conditions, representing the measured and/or observed normal or baseline movements and/or behaviors of, for example, a PUM that enables the one or more monitoring systems, including care hubs and/or care processing, to identity, at least in part, any deviations from this normal and/or baseline/control movements, patterns and/or behaviors. In some embodiments such movement sets can form part of a quiescent state of the SEE and any stakeholders therein. These quiescent states can form control or baseline states, where the PUM undertakes activities that are within thresholds of those states. For example, a control state for meals can include their preparation and/or delivery, consumption and removal or cleaning of any remains, cutlery or other aspects of the meal.

These deviations can then, in some embodiments, be represented, for example, as high dimensional representations, including vectors, topological representations and/or other representations that can be used by one or more other systems, including game theory, digital twins, AI/ML models, and/or other predictive systems to determine at the earliest possible time, the predicted and/or possible outcomes that can have an effect on the health, wellness, safety and/or care of the observed and monitored stakeholder, such as a PUM.

In some embodiments the measurements of the one or more sensors, devices and/or systems of the SEE, including those of any worn or carried devices can be communicated to one or more digital twins of that environment and/or the PUM therein, including any PPE of that PUM. For example, in many circumstances measurements of Pum are made on a periodic basis, for example weight when attending a health professional, and yet there may be variations in this and other measured physical characteristics of a PUM, which when communicated to a digital twin and correlated to the PPE and any other representations of a PUM, may provide indications of current and/or future health, wellness and/or safety events that would not have been identified otherwise. The use of digital twins for this continuous monitoring of the physical characteristics of a PUM can be undertaken in a manner that protects the privacy of the PUM, through for example use of tokens and cryptographic encoding of the digital twins and/or the data sets thereof.

### A Model for Motion

In some embodiments, one or more AI/ML models created using embeddings and an architecture such as transformers, configured for analyzing human body movements, in whole or in part, through for example one or more data sets generated by one or more sensors, devices and/or systems, present in a SEE which can be applied in various situations such as for example, detecting movements, patterns, behaviors, intentions, identifying, at least in part, health anomalies, and the like.

For example, a PUM who is present in a SEE can have their movements represented by the one or more data sets generated based on measurements by the one or more sensors, devices and/or system present in the SEE. This can include embedded, worn and/or carried, active and/or passive sensors, devices and/or systems in any arrangement.

In some embodiments, such an approach can include input from a set of sensors capable of detecting movement, devices and/or systems that can capture, in whole or in part, movement of individual limbs, individual joints, combinations of limbs and joints, full body movements and/or changes in body position and/or location in any arrangement. The set of movement detection capable sensors, devices and/or systems can include wearable ones, such as accelerometers, compasses, gyroscopes, altimeters and/or tension/pressure transducers. It can also include non-wearable environmental sensors such as cameras, mmWave radars, LiDARs and/or ultrasonic sonars and/or other active or passive sensing capable, sensors, devices and/or systems.

In some embodiments one or more data sets, generated by the one or more sensors, devices and/or systems of the SEE, including worn and/or carried devices and sensors, can have one or more labels paired with identified elements of such data set. For example, if the data set comprises an image, a label can represent and/or describe the image. In another example, however the data set comprises a stream of data, the identified elements may consist of a set of elements differentiated by time. However, in many circumstances one or more evaluation techniques, for example an AI/ML models and/or one or more filtering systems and/or characteristics of the data set can be used to identify the element, which can have one or more labels that represent and/or describe the element.

For example, a set of sensors, including an image sensor, microphone and mm RADAR generate a set of data occurring within a time period, that when evaluated determines that an event has occurred, and as such this event can be labeled as, for example a fall, trip or other PUM event.

Data sets generated by SEE/Digital Twin, AI/ML model can include real world physical measurements and/or synthetic data sets in any arrangement.

The use of labeled datasets from the set of sensors, devices and/or systems of a SEE to train an AI model to create embeddings can be used for an ML-like system that “understands” a person's movements in an environment, and can identify for example, typical expected movements, those movements with an increased risk and/or emergency-related movements, such as falls or collapses.

One aspect of using such a configured AI/ML model techniques for motion capture, evaluation, measurement and/or prediction is the consideration of the three-dimensional physical space in which such motion occurs, the fourth temporal dimension and other measured dimensions such as force, intention, velocity, acceleration, and the like. Many existing AI/ML models have little or limited integration of time and other dimensions and as language models, spatial considerations are generally limited to linguistic interpretations rather than measurements of the physical space.

One dimension that can be evaluated is the intent of the person who is exhibiting the movements, for example those measured by the one or more sensors, devices and/or systems present in a SEE. Such a dimension, or set thereof, can be explicit, in that the person may state their intent and/or in part or in whole hidden, in that the intention of the person is not explicitly revealed unless and until they have undertaken a movement or sequence thereof.

The separation of a series of incremental movements of one or more limbs and/or other body parts into quantified movement modules suitable for ingestion and/or processing by one or more AI/ML models can involve the use of multi-dimensional representations of these measured elements, for example represented by one or more languages, such as for example a specialized movement language.

In many current AI/ML models, tiling can be used to represent two dimensional arrays, whereas in the three dimensional space, that space may be represented as a set of cubes, where the dimensions of the cubes, in some embodiments, can be configured and aligned to the capabilities of the one or more sensors, devices and/or systems present in the SEE. Such an approach can include quantization, where, for example, the resolution of the measurements of the one or more sensors, devices and/or systems may be quantized to the dimensions of the one or more cubes and the contents thereof which the one or more sensors, devices and/or systems are measuring. For example, if a person is sitting, for example at a table and eating, then the granularity of the cubes used to measure the space, and the contents thereof may be configured to represent the physical attributes of that undertaking. For example, measurements from the one or more sensors, devices and/or systems can be represented as integers, for example in 4 or 8 bits, which can reduce the computational overhead involved in real-time or near real-time representation of the movements of one or more person in a SEE.

In some embodiments, an AI/ML models can employ, for example, linear time sequence, using a state space approach. The use of movement modules representing the movements of a PUM in the form of a sequence, for example those of a movement framework, a pattern and/or a behavior, can include the use of sequence models and structured state sequence models (SSMs), which are founded in the deep learning models, related to Recurrent Neural networks (RNNs) and convolutional neural networks (CNNs) in combination with state space models.

A PUM's movement data can be captured using a sensor set, for example as deployed and operating in a SEE, which can contain one or more combinations of wearable sensors, such as, accelerometers, gyroscopes, compasses, altimeters, and/or pressure transducers to capture individual limb and joint movements, as well as overall body changes, with non-wearable sensors, such as cameras, mmWave radars, LiDARs, microphones, and/or ultrasonic sonars to provide a broader view of the environment and/or track full-body movement, position and/or location. Data from these different sensors can be combined to create a broader and deeper representation of the movement of a PUM or other stakeholder using techniques like, for example, Kalman filtering and/or particle filters to correct for sensor noise and inaccuracies.

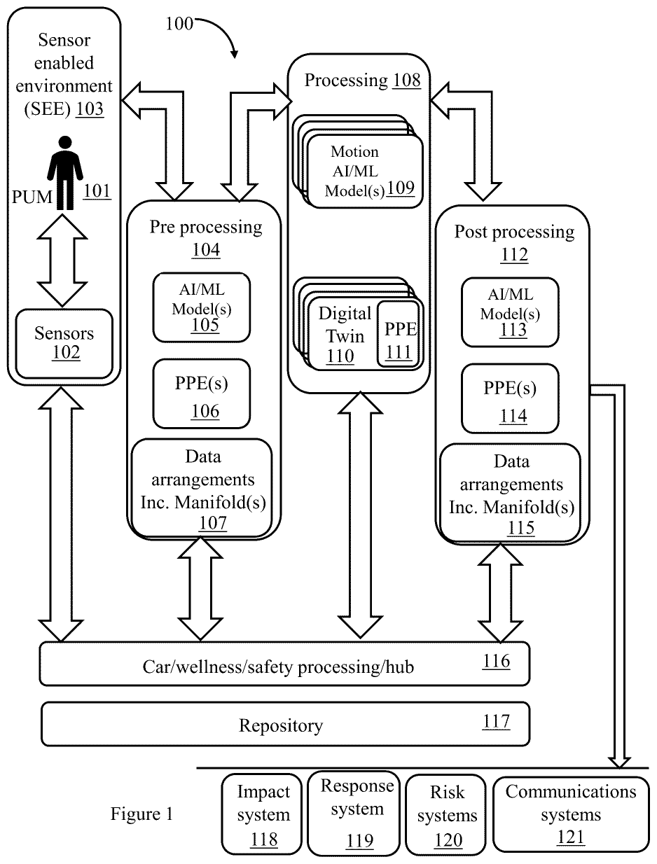

FIG. 1 illustrates an example system 100 for analyzing motion in a SEE, according to an example embodiment of the present disclosure. The system 100 employs a motion model 109. A PUM 101, monitored by a set of sensors, devices and/or systems 102 in a sensor enabled environment 103 is present. The data sets generated by the sensors, devices and/or systems 102 can be communicated to one or more pre-processing modules 104 and/or one or more care, wellness and/or safety processing systems and/or care processing systems 116. Such pre-processing module 104 can include one or more AI/ML models 105, Personal Physics Engines 106 and/or one or more data arrangements, including for example one or more manifolds 107. The pre-processing module may for the data sets from sensors 102 into one or more formats suitable for a processing system 108, which can include one or more motion models (e.g., AI/ML models) and/or digital twins 110 which can employ one or more PPE 111. The data sets generated by such processing systems 108 can undergo further processing, for example through a post processing module 112. Such post processing module 112 can include one or more ML 113, one or more PPE 115, one or more data arrangements, including for example manifolds, in any arrangement. The data sets, in part or in whole in any arrangement may be communicated by sensors 102, pre-processing 104, processing 108 and/or post processing 112 with the care processing systems 116, where one or more repository 117 can be employed directly or indirectly. The output of the pre-processing 104, processing 108, post-processing 112 and/or care processing systems 116 can be communicated to one or more further systems, including for example, impact systems 118, response systems 119, risk systems 120 and/or communications systems 121 in any arrangement.

### Data Set Preparation

In some embodiments, the data sets generated by the one or more sensors, devices and/or systems present in a SEE include data from measurements, where for example each of these sensors, devices, or systems provides data in the format for which it is configured. These data sets can form patterns, which may form further data sets generated by the one or more sensors, devices and/or systems and/or may be generated by, for example, a care hub or care processing system configured to do so.

In these examples, these data sets may be in form of tokens, where for example each of these tokens can include some form of identity that can, in some embodiments, be used to determine, at least in part, what other sensors, devices and/or systems may access and/or interact with the data of the token in whole or in part.

In some embodiments, this can include multi segment tokens, where the token may have multiple segments, each of which includes a payload and a set of meta data and/or governance controls for access and deployment of the payload of that segment.

In some embodiments, data sets from the one or more sensors, devices and/or systems present in the SEE, may be communicated to, for example a PPE and/or AI/ML model in any arrangement, in the form of tokens, where such tokens can only be accessed by authorized and/or authenticated systems. In some embodiments, the vectors, contextual vectors and/or other data sets may be communicated in the form of secure tokens.

The use of tokens can include the protection of the privacy of the PUM, for example to manage any PII (personal Identifying Information), such that only those authenticated and authorized entities, including stakeholders and/or systems are able to access such data for periods of time which are specified.

In some embodiments, these tokens, and/or data sets they represent may be converted into segments suitable for ingestion by one or more AI/ML model. This can include quantization such that the segments can form a set of tokens, which can include, for example, trainable weight matrixes that include keys, vectors and input tokens, that can be ingested and operated upon by one or more AI/ML model that is configured to do.

In some embodiments, such data sets may be prepared by, for example one or more sensors, devices and/or systems in any arrangement. For example, active sensors such as a millimeter radar may be configured to generate data sets based on movements, which can include lack thereof, of a stakeholder. This stakeholder may also have data sets representing their movements from, for example, other sensors, devices and/or systems, such as microphones, including mems microphones, haptic sensors, including strain gauges, cameras and/or other active and/or passive sensors, devices and/or systems. These data sets can include recognition of movements, where for example a stakeholder extends a limb, stands or sits, and as such the data sets of the one or more sensors, devices and/or systems can be combined to create, in whole or in part, an aggregated data set representing that movement, for example as a movement module. This data set may then be evaluated, including using one or more matching services, to determine, at least in part, whether this movement matches a previously known movement, which for example may be represented by a movement module, movement framework comprising a sequence of movement modules and/or a pattern comprising movement modules and/or movement frameworks in any arrangement. This data set can then be formatted to be used by, for example AI/ML models, where for example the preparation of the data can be in the form of a quantized element, for example a word, combination of words, sentence or other arrangement that matches those AI/ML models, and in some embodiments can form part of a language of movement.

One aspect of data preparation can be the use of context, for example represented by vectors or other high dimensional representations, which can form enriched embedding vectors, for example as part of a self-attention schema. This can include the data sets of the one or more sensors, devices and/or systems, that for example form a set of such present in a SEE, providing a set of input vectors, for example comprising the measurements of the sensor, device and/or system and/or other contextual data, such as the relationship of the sensor, device and/or system to other such sensors, devices and/or systems present in the SEE, which can include location, time, force, order, external conditions, for example temperature, humidity or other environmental considerations and/or data sets measuring the physiological attributes of a stakeholder. In some embodiments, these data sets, represented by one or more vectors can form one or more contextual vectors, which can include differing arrangements of such input vectors representing these data sets and/or one or more weightings of these vectors, to generate one or more contextual vectors.

In some embodiments a set of measurements comprising the context of the SEE and/or the PUM therein, can for example, be represented as contextual dimensions, for example as contextual vectors which may be employed to generate a set of self-attentions, where for example depending on the weights applied and their relative values, order and/or importance to each other, differing context dimension values may be calculated based on the same input vectors. This can form a set of alternative perspectives on the monitoring of the one or more stakeholders present in a SEE, for example the PUM, such that when provided to an AI/ML model, and for example employing one or more digital twins, differing predicted outcomes may be generated. For example, each of these outcomes can represent a differing movement, including sequences thereof, for a stakeholder, for example a PUM.

In this example, each of these outcomes can be, for example, provided as input to a PPE, where they can be compared with the possible and/or historical movements of the PUM, for example using previous movement patterns and/or behaviors to determine, at least in part, the movement that is being undertaken by the stakeholder and/or the most likely movements that such stakeholder can undertake. In some embodiments, this can include prediction, identification and/or determination of the intent of such movement.

In some embodiments, an AI/ML model may be employed during the initialization, calibration and/or configuration of a SEE to determine, at least in part, the relationships between the one or more sensors, devices and/or systems of that SEE. This can include developing one or more models where the sensor, device and/or system and the data generated by them can form differing weights and/or contexts dependent in part on the data generated by a specific sensor, device and/or system at a time, which can represent a movement of a stakeholder, for example the PUM, in an environment. For example, such models may include contextual vectors which can be used to train one or more other LLM, SLM and/or provide vectors to an embedding.

In some embodiments, context vectors may be input to a PPE, where they can be evaluated. Those context vectors that represent movements that are outside of the capability of the PUM, such as jumping or climbing when the PUM, for example has bad knees, may be communicated to one or more monitoring systems and can result in testing, calibration and/or configuration of the sensors, devices and/or systems that were involved in the measurements and/or if the source was, for example, one or more AI/ML models, the PPE operate as guard rails, for example as a RAG to those AI/ML models, including and communicating the impossibility of such movement. In another example, those movements that are within one or more thresholds, for example twitching of an eye, hand or other minor affliction, such that these movements and the contextual vectors in which they are represented can be removed from any data set provided to an AI/ML model.

In some embodiments, these data sets from the one or more sensors, devices and/or systems present in the SEE, may be communicated to, for example a PPE and/or AI/ML model in any arrangement, in the form of tokens, where such tokens can only be accessed by authorized and/or authenticated systems. In some embodiments, the vectors, contextual vectors and/or other data sets may be communicated in the form of secure tokens. In some embodiments, one or more certification process, for example using encryption, can be employed for the data sets generated in whole or in part by the SEE and/or data sets generated by the carried/worn sensors.

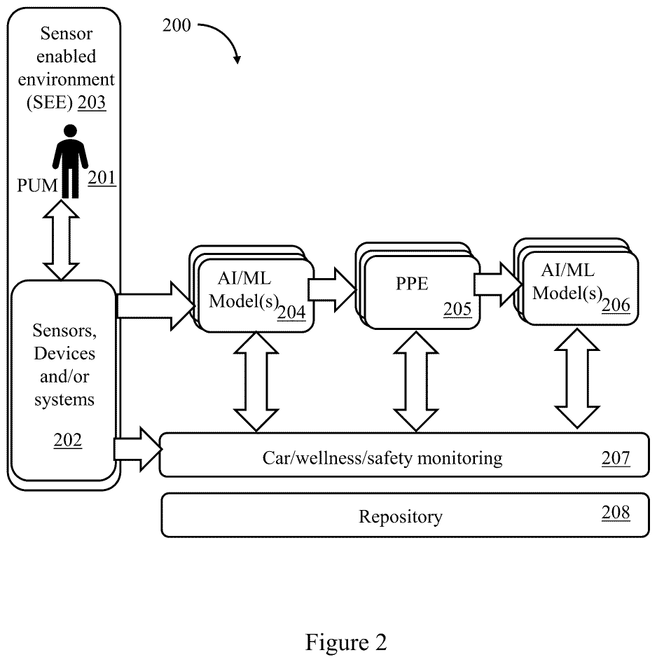

FIG. 2 illustrates an example system 200 which incorporates a first stage AI/ML model 204, a personalized physics engine 205, and one or more additional AI/ML model 206 in a multi-stage processing architecture, according to example embodiments of the present disclosure. a PUM 201 is present in a SEE 203, which includes sensors, devices and/or systems 202 that are employed, at least in part, to monitor the PUM 201 and the SEE 203. The data sets generated by the sensors 202 can be communicated to one or more first stage AI/ML model 204, which can be configured to process such data sets, for example by identification of movements of a PUM or by performing other filtering actions on the original data sets. This can include the use of motion LLMs and more general AI/ML modules and the elements thereof in any arrangement. The data sets generated by the first stage AI/ML model 204 can be communicated to one or more PPE 205, where for example, the output thereof may be communicated to one or more additional AI/ML models 206, which may include motion LLM and/or other LLM configured for specialized operations. The sensors, 202, first stage AI/ML model 204, PPE 205 and/or additional AI/ML models 206 may communicate with one or more care, wellness and/or safety monitoring system 207, all of which may interact with one or more repository 208 in any arrangement.

### Embeddings: Body Part Movements Representation

Embeddings can be dense vector representations of data points, for example, like words, sentences, in the case of natural language processing (NLP) and LLMs, or images, in the case of multi-modal LLMs. These dense vector representations can be transformations of, for example, any audio, image and/or video, haptic, LIDAR, RADAR, temperature, humidity and/or other environmental factors and/or other measured data provided by the one or more sensors, devices and/or systems of a SEE. Each vector in a dimension space can represent one or more specific feature or aspect of the data. Embeddings can capture semantic relationships, for example, words with similar meanings can have vectors closer together in the embedding space. Similarly, data sets with similar attributes, for example those within a specific timeframe, from the same location, including certain consistent data may have vectors that are close together and as such may be bundled. A common technique for converting data records to their embedding representation is to use a pre-trained AI/ML model.

Embeddings for movement sensor data can be done for individual sensor data records, using vectors that represent, for example, each possible movement of an individual joint. Other embedding approaches can associate each vector to a sequence of sensor records (sequence of movements, including movement modules, of different joints and locations of limbs) to represent, for example, possible full complex movements of the full body. Some approaches can include context labels to the embedding model generation, in order to have such context represented in one or more dimensions of the embedding vectors.

One or more AI/ML models can be trained on a labeled dataset, optimizing the AI/ML model(s) to generate embeddings that effectively represent different movements, sequences of movement and/or movement categories in the different contexts of interest where they can occur. After initial training, the embedding models can be fine-tuned on a specific individual and/or PUM category, using training datasets that cover their particular movement and/or pattern and/or behavior cases.

For example, data sets generated by the one or more sensors, devices and/or systems of the SEE, including for example those that have been identified, and potentially classified, as movement modules, movement frameworks, patterns and/or behaviors, can be used to label these data sets as such. In this example the AI/ML models can be configured to operate on these labeled data sets, for example a PPE that includes such a configured AI/ML models may use the model trained on such data to predict the movements of a PUM and/or to validate and/or verify that the predicted movement is within the physical capabilities of that PUM. Such an approach can include the use of one or more digital twins by and for a PPE in the prediction, calculation, verification and/or validation of such movements.

In some embodiments, a labeled dataset for training an embedding model, and/or other AI models, can comprise a plurality of digital data samples, each associated with one or more semantic labels expressed, for example, in natural language, wherein the labels guide the model to generate vector embeddings that encode perceptual and contextual similarity and are interoperable with large language models for retrieval and reasoning tasks.

A labeled dataset comprises input data elements (e.g., images, audio signals, text sequences, sensor measurements), and corresponding labels, where each label encodes semantic, categorical, numerical, or relational information describing the associated input data element. Using such datasets to train machine-learning models enables the models to learn a mapping between input representations and the labeled information through supervised or weakly supervised learning processes. For example, in some embodiments, a labeled audio/sound dataset can be created to train an audio embedding model whose embeddings can be indexed in a vector database, and queried or reasoned over by an AI/ML model, for sound search, classification, and/or multimodal reasoning, as part of an health, wellness and/or safety event detection, including emergency event detection and/or confirmation process. This sound dataset can be created by defining the audio domain (e.g. environmental sounds, human-made sounds), then collecting raw audio samples, from, for example, field recordings, public datasets and/or synthetic generation. The audio samples can then standardize, for example using technical characteristics, such as format, sample rate, bit depth, duration, etc., and defining a label schema, which can include categories, descriptions, attributes, and/or confidence level, on one or more categories. Then one or more labels can be assigned to each sample. After this, the samples can be converted to model-ready features, by extracting numerical representations of the samples, such as Mel spectrograms, Log-mel filter banks, raw waveform segments, etc. These data representations, and their labels, can be used to train an AI model for, for example, embedding, allowing the model to map each audio clip to a fixed length embedding vector that captures semantic meaning.

In some embodiments data from other sensors, such as, for example, mmWave radars, cameras, WiFi signal-based location sensors, wearable accelerometers, gyroscopes, altimeters and/or other body part movement/position sensors, etc., can be used in a similar manner, using single-sensor samples and/or multi-sensor data samples, to generate labeled datasets and train AI models with them, for embedding and/or other purposes.

In some embodiments the data samples and/or the labels can include contextual information, such as, for example, time of day, location, person's intent, etc., which allows the trained model to map sensor data plus context to a semantic meaning in the form of an embedding vector. In some cases the contextual information can be provided as a vector, resulting from a pre-existing embedding model. For example, in some embodiments, an AI/ML models can learn to represent body part movements as numerical vectors, similar to words in a traditional NLP model. These vectors can be generated using different embedding techniques, including but not limited to:

Convolutional Neural Network (CNN): For example, this technique, which is currently commonly used for image processing, can be applied to movement data. CNN's can employ feature mappings, which in some embodiments can include movement modules, patterns and/or behaviors, including those employed as part of a PPE, which can include physical aspects. In some embodiments, a CNN may employ cubes as part of the feature detection of movements of a PUM, where the granularity of the feature detector may be dynamically adjusted, based on the movement being evaluated.

Recurrent Neural Network (RNN): For example, RNNs are well-suited for sequential data sets, such as sequences of data sets, patterns and/or behaviors and/or combinations of body part movements, for example limbs, including segments thereof, such as fingers, wrists, elbows and/or shoulders. The use of RNN's to represent the context, in for example temporal terms, of a movement can support the prediction of future movements based on previous movements and/or those movements currently occurring, including those that are possible, for example those represented by a personalized PPE. For example, this can be applied to movements, such of those of the hand where there is a sequence, for example when picking up an object or carrying an object there can be a specific set of movements that are carried out in a sequence, which can, for example include wrists, elbow and/or shoulder or other joints in one or more arrangements. These movement sequences can, for example, be represented by one or more computation graph, which can form part of a PPE and/or AI/ML model.

In some embodiments combinations of CNN and RNN techniques can be employed to, for example, to represent linear time sequences, using a state space approach.

Graph-Based Embeddings: These embeddings can employ a graph structure, including for example graph databases, to represent, at least in part one or more relationships between body parts and the movements thereof. This can include one more computational graphs, for example directed acyclic graphs.

Topological representations: These embeddings can include topological representations of data sets in the form, for example, of a manifold or other topological representation. For example, this can include Hilbert transforms, to represent co-dependent relationships.

In some embodiments, movements of a PUM within a SEE may be represented in the form of manifolds, such as Riemannian manifolds and the like. These representations can then be used to, at least in part, format the data sets generated by the one or more sensors, devices and/or systems present in the SEE, to generate a set of vectors and/or weights that can be employed by, for example, embeddings and/or transformers of one or more AI/ML models. These manifold representations can also be deployed as part of one or more PPE, for example representing neighborhoods into which the PUM, including parts thereof, may move into. For example, in some embodiments, the body of the PUM and the limbs and joints thereof may have a neighborhood in Euclidean space where the PUM body, including parts thereof, for example an arm, leg, hand and the like may move to. These neighborhoods may be diffeomorphic in that the vectors of the direction and/or force of the movements of the PUM can be considered as functions that are continuously differentiable.

In some embodiments, vector bundles can be employed to group together sets of vectors that, for example, have been generated by an AI/ML model including those forming part of an embedding process, where these bundles can form a smooth manifold, that represents, at least in part the direction, force and/or timing of the movement of one or more body parts of a PUM.

Geometric reasoning: The movements of a person can be represented as a set of geometric movements of the human body. For example, a rotation of a human neck cannot be 360 degrees, nor can a functioning human knee joint be bent forward to over 90 degrees. These movements can form part of the geometry of a human body, which can be represented, in some embodiments, in terms of the range of motions that a specific body part, set of body parts, such as a limb, and/or sequence of body parts can undertake.

These fundamental geometries of movement can be particularized for one or more stakeholder, as even with the differences across body types, shapes and conditions, certain geometries are common to the human physical form. These geometries may form, at least in part, a classifier that can be deployed as part of an AI/ML model. In some embodiments a PPE may include a muscular skeletal model of the human body that incorporates these geometries of movements and can form a reference for the creation of a personalized physical model of a PUM which forms part of a PPE.

Such an approach can form a set of properties, at least in part, of a physics engine for a human, which can then be personalized for the specific human being monitored, such as a PUM. This can employ a set of geometric reasoning, which in some embodiments, can be coupled to, for example, one or more game theory engines and/or games to represent the potential movements of a PUM and/or determine the most likely behaviors such can movements form.

A further aspect of this geometric approach is the determination, at least in part, of the meaning of a movement, where for example, some movements may have no or little meaning, such as a hand idly brushing the face, resting the hands, crossing the legs and the like, whereas other movements may indicate attempts to undertake a movement, pattern and/or behavior, such as standing, walking reaching, operating an appliance and the like, which can have varying degrees of success and can have varying degrees of risk.

Geometric reasoning can also be employed to determine, at least in part, the potential interactions of the stakeholder with an environment, where the contact points can be identified. For example, if a stakeholder is walking there are the contact points for their feet as they traverse an environment. Additionally, if a wall or furniture is within reach of that stakeholder this can also represent contact points.

In some embodiments, the contact points and areas may include the calculation of those areas and points that a person may interact with, for example if they fell, tripped or otherwise moved in a manner that indicates a problem or issue with their balance.

In some embodiments, geometric reasoning and game theory may be combined to form, for example, a set of games where there are strategies involving the interaction of the stakeholder with the environment. For example, game theory games can include those where, for example a PPE and one or more digital twin can be invoked to represent the possible movements of a PUM, and the potential contact areas of that PUM with the environment. This can, in some embodiments, be used to ascertain the one or more strategies that can be employed by a PUM, for example those where the PUM mobility is impacted, such as after a procedure, such as knee, shoulder, hip or other surgery and/or when the mobility of the PUM is, over time, deteriorating. In this example, the game theory game and PPE combination may provide alternative strategies that enable the PUM to undertake the mobility behaviors in a manner that is suited to their current mobility circumstances. In some embodiments, these strategies can be made available to the PUM though for example carried, worn and/or embedded devices, through for example audio, video, text and/or haptic interactions. This approach can include communicating with other stakeholders, such as carers and/or neighbors to assist the PUM in undertaking such strategies.

Deep feed forward neural networks: This approach may be employed to overcome the limitations of linear models, for example those based on the geometry of the movements of a limb or part thereof, particularly when there are, for example multiple limb segments in action, for example fingers, hands, wrists and/or elbows, such as when lifting a cup, or placing an item on a surface.

In some embodiments, one or more mapping functions may be applied to inputs, such as for example data sets generated by the one or more sensors, devices and/or systems present in a SEE. This approach can employ one or more deep forward networks. In this manner general movements and/or combinations thereof can be generalized to form an initial mapping that can, for example, inform AI/ML models including those employed by and/or form part of a PPE.

One application of this approach can include the transformation of vector spaces, for example a 3D environment in which a PUM is making a movement and the scalar for the force exerted by that movement, both in terms of the force expended by the PUM, for example the muscular force and the force exerted on an item, surface and/or object involved in the movement and the physical dimensions and geometry of that movement. For example, such transformations can use, for example, rectified linear unit (ReLu), where an activation function, for example one detected by the one or more sensors, devices and/or systems as an event, including a change of state, such that the linear function, for example using geometric evaluations of a 3D Euclidian space can be transformed into data sets suitable for training a neural network to, for example, enable gradient based learning.

In some embodiments, the cost function of the neural network may be correlated to the energy exerted by a PUM in undertaking one or more movements. The determination of this cost can involve, for example, approximations and/or predictions based, at least in part on the movement itself and/or the condition of the PUM, including for example using an energy “budget” representing their total available energy for a period of time, for example an hour, day, minute and the like, such that each movement exerted force expends a certain quanta of the energy budget, for example expressed in joules or other energy metrics.

In some embodiments, the energy expended by a PUM may be communicated to them and/or to other stakeholders, including carers and/or other systems monitoring the PUM, such that the PUM and/or other stakeholders may be advised of the energy expended by the PUM and/or predicted to be expended by the PUM. This can include measurements and/or calculations of the total energy available to a PUM, such that if the PUM is approaching the level of exhaustion of their available energy, with the commensurate increate on risks as they undertake any tasks, can be identified and communicated to the PUM and/or other stakeholders to avoid, and/or mitigate any potential wellness, heath and/or safety impact on the PUM.

In some embodiments, the measurement of the recurrent behaviors, including patterns, in terms of the movements, which can include movement modules, time, force and/or energy expenditure can indicate any deterioration of PUM capabilities. Energy can be calculated by, for example using a PPE and/or one or more AI/ML models to determine through for example, prediction, estimation, measurement and/or other calculation the energy expended for one or more movements by a PUM. This can include the time taken for a PUM to undertake a movement, which can be part of, for example one or more movement sequence, pattern and/or behavior. This can also include the force expended by a PUM in undertaking such movements, for example as measured directly and/or indirectly through the one or more sensors, devices and systems present in a SEE, including those worn or carried by the PUM.

For example, calculation of PUM energy levels can include monitoring of diet, waste and/or exercise to calculate the available energy for a PUM at or over a period of time. Factors such as Time of Day, restfulness, mental resilience, patterns and behaviors can be used as part of the calculations. In some embodiments an AI/ML model can be employed to generate a model of energy of a PUM, for example in a digital twin, based on the movements, activities, events, patterns and behaviors observed and measured by the SEE.

In another example if the PUM is undertaking an exercise or other activity, such as walking, then measuring of their movements, using one or more sensors, devices and/or systems, including those worn and/or carried, can provide datasets that, at least in part, indicate tiredness, reduction in function of the activity and/or other indicators of energy expenditure. These datasets and/or indicators can have one or more risk metrics that can be bound to the activities, potentially on a sliding scale, for example as tiredness increases so does the risk metrics. In some embodiments, these risk metrics can be output by one or more AI/ML model in an arrangement with a PPE. These risk metrics can be communicated to one or more monitoring system, including care hub and/or care processing and/or to one or more stakeholders, directly and/or indirectly. For example, a risk metric indicating that the PUM is nearing exhaustion or other significant tiredness, may be communicated to a monitoring system, which in some embodiments can generate a communication, for example using audio, video, text, haptic of other forms of message, to advise the PUM of the increased risk and one or more mitigations that they can undertake. In this example the monitoring may also create communications with other stakeholders, for example, a career.

One aspect of determining the relative movement capabilities of a PUM, including their ability to undertake an activity, can be based on measurements, for example those undertaken by the one or more sensors, devices and/or system of the SEE, including those worn, carried and/or embedded to determine, in part or in whole, the muscular strength, bone density, agility, flexibility and/or other physical attributes of the stakeholder. These attributes can be represented by one or more PPE and can, in some embodiments, be used by one or more AI/ML models, predict the likely state of the PUM at the commencement, during and/or after their undertaking one or more activity. These predictions can inform one or more monitoring system as to the state of the PUM such that the monitoring system, which can include one or more risk evaluation systems and/or configurations, can invoke one or more response systems, to, for example, communicate with the PUM, another stakeholder. This can include varying the configurations of the one or more sensors, devices and/or systems that are monitoring the PUM to align with the activities and risk metrics thereof, for example this can include increasing the sensitivity of a microphone to better determine the PUM's breathing during an activity and/or using other sensors, monitor the oxygen/blood ratio during exercise or other activities. These increased monitoring capabilities can inform one or more response systems resulting in one or more communications to the PUM and/or other stakeholders, prior to, for example, a predicted care, wellness, health and/or safety event.

In some embodiments, the movements of a stakeholder, including a PUM, can form part of a game theory game that represents those movements, for example where each limb and/or joint is a player in the game and the payoffs of the game represent movement combinations, including sequences thereof, that match the observed and/or measured behaviors, including patterns thereof, of one or more stakeholder present in a SEE.

These games can, in some embodiments, form hierarchies or other arrangements of games where various sub games can represent the movements of one or more limbs and/or joints and the overall game can, at least in part, represent the movements of a stakeholder. This can, for example, include anticipated and/or predicted movements, where for example the PPE, generates a set of anticipated movements for a limb, joint, combination thereof and/or a behavior of a stakeholder and such predicted or anticipated movement forms part of the game, for example as another player, where the payoff is matched to the observed behaviors, including previous behaviors matching, at least in part, these movements sets. In this manner the strategies of the movements of a stakeholder, including the PUM may be aligned to their behaviors to determine at the earliest possible time any actual or potential impact on the wellbeing, health, care and/or safety of the PUM and/or any other stakeholder.

### Transformers: Movement Analysis and Understanding

In some embodiments, an AI/ML model, such as an LLM, based, for example, on a Transformer architecture, can be trained using movement embeddings datasets, generated by one or more sensors, devices and/or systems comprising the SEE, which are enhanced and/or labeled based on the training objectives, such as identifying typical and/or expected movements, recognition of movements with risk metrics, such as those determined to be a risk to the health, wellness and/or safety of a stakeholder, and/or detection of situations that require an immediate response, such as an emergency. In some examples, the resulting AI/ML model can be fine-tuned for particular individuals, individual categories and/or recognition and/or inference targets.

Further fine-tuning can be done on the AI/ML model using, for example, a dataset of text descriptions paired with corresponding movement embeddings to specialize the AI/ML model to associate specific movements with textual representations. Such dataset descriptors, which can be human and/or machine interpretable, can be employed by one or PPE and can, in some embodiments, form part of the configuration of such PPE.

In some embodiments this can include descriptions of movements, including sequences, of one or more stakeholders, including the PUM. These descriptors can be at multiple granularities, for example, from a single joint such as in a finger, to a behavior, for example going to the bathroom. These descriptions may be in the form of human readable text, where such text can describe the particular movement of a PUM, for example a pattern or behavior. In some embodiments, an LLM may generate, potentially in collaboration with AI/ML model including agents, a description of the movement of the PUM, which can be human and/or machine interpretable.

In some embodiments a PPE may be configured to represent movements, including patterns and behaviors, where these movements have attributes that can include descriptions and/or other data sets. For example, this can include movement descriptions, such as “reaching for an object” and/or intentional attributes, such as “make coffee”, “drink coffee” and the like.

The correlation of a model to the relative granularity of a set of movements of, for example, a PUM can employ a PPE, where the PPE includes a set of frameworks of the possible actions of a human muscular/skeletal body at differing granularities. For example, the PPE can have a framework for a hand, which can represent movements of the fingers and opposing thumb, including the force that can be exerted by a hand. These aligned movements can, at least in part, inform one or more monitoring systems, for example those of a SEE for monitoring a PUM. These communications can be used to imitate one or more responses for and to a PUM and/or other stakeholder, including providing data sets to one or more risk modules.

This can form part of a model deployed and/or operated by, for example an LLM and/or other AI/ML model, where this model is then populated by data sets from the one or more sensors, devices and/or systems, which in this example, can include sensors that are, for example, worn on the hands, either temporarily to gain such measurements or permanent, such as a ring, bracelet or other worn set of sensing. This can include, for example, clothing or other worn elements that include sensing, such as skin, moisture, heat, chemical, tensile, haptic sensors and the like.

Such frameworks and subsequent models can then form part of a further framework and/or model, for example an arm, where each of the joints and the relationships between them are conformant to the overall muscular/skeletal model represented in the PPE, with the relevant force, velocity, acceleration, rotation and other physical attributes embedded therein.

The set of frameworks and/or models can be combined to, at least in part, represent the movements of a PUM in a SEE as they undertake their daily activities, where for example an AI/ML model may use such a model to predict the behaviors of the PUM in an environment where a care monitoring system, for example care hub and/or care processing system can then be used, for example in collaboration with a PPE, digital twins and/or AI/ML model to, based on the data sets generated by the one or more sensors, devices and/or systems of the SEE, determine any variations between the predicted and actual behaviors. These variations can, in some embodiments be used to fine tune the models employed by the AI/ML model and may be modulated by the overall state of the PUM and/or the SEE, such that if the state is quiescent, then one or more AI/ML models may be employed to tune the model, whereas if the state includes one or more events, actions, state changes or other data sets that is evaluated to exceed the thresholds or other metrics for a quiescent state, then this data can be used by the care monitoring system to initiate and/or undertake one or more responses. For example, if a PUM is having difficulty rising from a sitting to standing position, for example as indicated by their movements, the monitoring systems may advise the PUM to remain sitting and/or notify one or more other stakeholder, such as a carer to assist the PUM.

In some embodiments, the AI/ML model employs transformers and/or other modules to operate on sequences of natural language units, where such sequences are composed of words, including sub words. This can include use of pretraining and can be employed for any of the one or more AI/ML models employed and/or for determining, at least in part, the weights that presented to an attention mechanism.

In some example embodiments this approach can be employed for operations on sequences of data sets that are quantized and segmented, for example movement data sets, into a format that is suitable for an ML, including transformers and/or other modules to operate upon. For example, this can include specifically designed AI/ML model for example motion LLM/SLM where the modules thereof for sequence-to-sequence movement analysis tasks, which can be in the form of frameworks, and can include;

Encoder-Decoder Architecture: The encoder takes in a sequence of movements (e.g., joint positions and transitions) and produce a representation that can be continuous.

Self-Attention Mechanism: This mechanism allows the model to focus on specific parts of the input sequence, enabling better understanding of complex movement patterns.

Specialist transformers: Can be employed for AI/ML model (e.g., LLM, SLM) and movements in multi-dimensional spaces.

Specialized attention modules: Can include both static and dynamic weights.

Tokens: that represent movement expressed for a language traditional transformer.

Token sets: for embedding, which can include pre-weighted data sets.

Multi-modal tokens: that can use text for context, text tokens for description of movement, tokenized spatial representations (including temporal).

Arbitrary mixes of measurements and/or representations including languages.

Combinations of PPE/LLM/SLM/Neural networks and/or other AI/ML models.

Validations and/or sequences of use and/or baseline/control cases for variations, including physics informed neural networks.

### Training Data: Body Part Movements

In some embodiments, training a motion LLM, SLM and/or other AI/ML models can include one or more data sets, for example those of the one or more sensors, devices and/or systems present in a SEE, where for example such data sets can be provided as training data in multiple formats, including annotated, categorized, forming patterns, in whole or in part, quantized as movement modules, represented as and within frameworks of typical movements and/or other arrangements of movements and/or the like in any arrangement. For example, this can include:

Movement modules: A dataset consisting of human movement sequences, focused on distinct body parts (e.g., joints, limbs), for example an arm and the set of movements that the joints and limb may undertake.

Movement Frameworks: A movement framework can comprise one or more movement modules in a sequence, for example a movement framework may be an arm, comprising movement modules for hand and wrist, forearm and elbow and upper and shoulder.

Labels or Annotations: Relevant labels, such as intentions, emotions, or health status, represented, for example, as attributes of each record in the movement modules dataset. This labeling can be created by a combination of human and/or automated data labeling.

Patterns: Sets of patterns, which for example, can comprise sets of movements, including for example movement modules and/or movement sequences.

Sequences: Arrangements of movements, including movement modules and/or movement frameworks, patterns and/or behaviors that can form, in whole or in part a sequence of such in an order, where such order can, in part or in whole, be represented in one or more PPE, including personalized PPE. In some embodiments, an generative AI system such as an LLM or other AI/ML models may be configured to use, at least in part, a recurrent neural network (RNN), where for example weights are shared for the one or more data sets generated by the one or more sensors, devices and/or systems present in a SEE.

One or more training data sets can be generated or created for AI/ML model training and/or validation, by collecting sensor, device and/or system set data from one or more individuals and their environments, including a SEE. One or more data collection sessions, which can be continuous or asynchronous, can be performed for each individual, for example a PUM, which in some embodiments, can include focusing on scenarios of interest for the AI/ML model training objectives, such as typical/expected movements (walking, running, sitting, standing, reaching, etc.), movements that can include a degree of risk, for example expressed as risk metrics, (awkward stances, sudden changes in direction, excessive leaning, etc.), emergency movements (falls, collapses, trips, etc.) and/or combinations of movements associated with, for example, common routines for different locations in the person's location and/or different times of the day, for example those representing the behaviors of a PUM. Further additional labels can add context such as intention of the movement.

These datasets can be enhanced, expanded and/or improved in other ways using synthetic sensor data generated by a physics engine, such as a PPE, and/or a body animation model configured to simulate the movements and scenarios of interest for the AI/ML training objectives. In some embodiments, for example, the accelerometers' output can be obtained by measuring linear acceleration in different axes (X, Y, Z) at the sensor's location on the simulated body. The Gyroscopes' output can be obtained by calculating angular velocity (rotation rate) around different axes of the limbs where the sensors are to be located. Tension/pressure transducers can be simulated by calculating forces exerted on specific parts of the body and/or the contact points of the environment exerted by the body for example when walking.

The datasets can be then labeled using label categories such as, for example:

Movement Classes: categories of movements for the AI/ML model to understand:

Typical/Expected Movements: Walking, running, sitting, standing, reaching.

Risky Movements: Awkward stances, sudden changes in direction, excessive leaning.

Emergency Movements: Falls, collapses, trips.

Other labels can be defined to differentiate activity context, such as, for example, time of the day, common activity (eating, cooking, watching TV, sleeping, walking the dog), person's context, such as health status (hip replaced, recent knee injury, heart condition, etc.), and/or other context categories.

Labeling Data can include a training dataset being annotated with the corresponding labels by, for example, comparing data sets generated by the one or more sensors, devices and/or systems of the SEE, including those data sets that have been stored in one or more repository, with previously measured data sets from the SEE, to determine which labels to apply to each dataset record. This can be done manually and automatically, using, for example an AI/ML model, such as, for example, a multi-modal LLM, or combining manual and automatic techniques.

In some embodiments, the training process involves optimizing the AI/ML model parameters to minimize the difference between predicted and actual outputs. This process can employ techniques such as loss functions like cross-entropy for classification tasks or mean squared error for regression tasks, and an optimizer, such as AdamW, to balance gradient descent and learning rate adaptation.

In some embodiments, multimodal fusion cab be employed to combine movement data with datasets from other modalities of sensors (e.g., sound, speech, facial expressions, mmWave radar and the like) for more accurate analysis and pattern identification and/or determination.

In some embodiments the training data sets for one or more AI/ML models that include, at least in part, personalization for one or more stakeholder, for example the PUM. This personalization can include, for example the movement artifacts, which can for example be removed from this training data, for example using a PPE configured for that purpose, movement modules, patterns and/or behaviors, including for example sequences thereof, which can form, for example data sets that are matched to the timing of their occurrence, for example, hourly, daily, weekly and the like in any arrangement and/or granularity of time. These training data may be used by a variety of AI/ML model, for example where such training data is anonymized and used, in part or in whole to generate an outcome for a diaspora that, for example shares certain common criteria, such as for example age, health or other condition, location and/or the like.

### Vectors and Higher Dimensional Spaces Including Manifolds

In some embodiments, one or more manifold can be employed as a higher dimensional representation of the movements, patterns and/or behaviors and their relationships to each other, of one or more stakeholder, including the PUM in a SEE. For example, in some embodiments, there can be data sets generated by the one or more sensors, devices and/or systems that include measurements in three physical dimensions, X, Y, Z representing Euclidean space, with the fourth dimension of time, which in some embodiments may include further dimensions such as measurement and/or calculation of force, for example including mass, acceleration and/or velocity.

In some embodiments, the recognition of the state of a PUM, for example the degree to which they have tensed their muscles as part of or in preparation for a movement can be represented by a neural model, that forms part of a PPE. For example, reaching to grab an object involves a set of muscles in the arm, wrist hand and upper body, which can be represented in the muscular skeletal model of the PUM. Such representation can use a digital twin and PPE to predict and/or project the trajectory, target and/or intention thereof, of the activity of the PUM. The actual physical action of the PUM as they undertake the movement, can be measured by the one or more sensors, devices and/or systems of the SEE and any difference between the digital twin version and the real time physical version can be identified. This can include the calculation of the movement of the muscles involved, including calculation of energy expended, degree of muscle contraction and the like, which can be used to inform the PUM or stakeholder of their condition. This can include identification of activities, for example exercises that can strengthen those muscles, where the muscles are identified as being weak or under-performing.

A PPE can include and/or employ, for example using one or more digital twins, one or more manifolds which can represent movement, gait, posture and/or other aspects of the PUM and/or other stakeholders. For example, many human movements can be represented as low dimensional manifolds, for example gait can be represented as a one-dimensional manifold which can be embedded in a high dimensional space, for example that generated by the measurements of the SEE and/or projected into one or more digital twin. For example, each of these data sets may be evaluated by one or more AI/ML model where the weights, attentions and/or vectors are determined by the AI/ML model. These can, in some embodiments, be represented as a manifold either prior to or after the AI/ML model operations, including embedding.

The relationship of one or more vectors ascertained by the AI/ML models to other vectors obtained in a similar manner and/or further vectors that are, for example, part of the data sets presented to the AI/ML model, can be represented by vector bundles. This approach can be used to determine, at least in part, the type and configuration of a manifold, which can represent the movements of a PUM, including parts thereof, including the number and types of dimensions forming such a manifold, as many movements can be represented by a set of vector bundles can form the dimensions of such a manifold. The intrinsic human movements generally have low intrinsic dimensionality, including when there is a movement with a clear intention, for example when a PUM picks up a cup, moves from sitting to standing and the like. In these examples, the human motion is smooth and constrained by the joint and other physical limitations of the body, which can be represented by a PPE. As such the PPE can be used to replicate a human movement, including a set thereof, and using one or more digital twins generate a sets of movements and the data sets thereof, including in the form of manifolds, that can form a training data set for one or more AI/ML models, such that possible movements of a PUM can be represented without requiring the PUM to physically undertake those movements.

In some embodiments, one or more sensors, devices and/or systems may generate data sets that, at least in part, represent those areas, surfaces and/or objects with which a PUM can come into contact. These contact areas may be mapped to, for example, provide an AI/ML model with contextual data. For example, if the AI/ML model is predicting movements, including using a PPE, of a PUM based on the data sets generated by the one or more sensors, devices and/or systems, this contextual data may be applied to constrain the possible predicted movements and/or to calculate the interactions of the PUM with such a context. For example, the AI/ML model may calculate the potential impact of a PUM moving their arm, where that arm contacts, for example a wall, which can both constrain the possible movement of that PUM and/or provide a calculation as to the impact of such a contact. This contact impact data can, for example, be provided to one or more other system, including risk evaluation modules and/or one or monitoring systems, such as care hub and care processing, which can then communicate with one or more response systems to initiate, for example an alert to the PUM and/o other stakeholders. In this example, the PUM contact with the wall may be to stabilize their movements and consequently the monitoring systems, using the one or more sensors, devices and/or systems present in the see, may ask the PUM “are you OK” or “do you need assistance” and/or may contact one or more other stakeholder, such as a carer, that the PUM is having difficulty.

In some embodiments, the one or more points of contact of the limb of a PUM, for example when the PUM is walking, can be predicted, using for example a PPE and one or more digital twins, such that any variations of these movements may be measured, using for example the known locations of the points of contact, for example those identified by a mapping of the environment. For example, AI/ML model, which can be in collaboration with a PPE, may predict the points of contact that a PUM will undertake as they traverse an environment, for example moving from a living area to a kitchen and the like. In this manner these predicted contact points can be communicated to the one or more sensors, devices and/or systems of the SEE so as to measure the conformance of the PUM movements with those predicted, such that any variations can be identified. These predictions can, in some embodiments, be used to calibrate and/or configure the one or more sensors, devices and/or systems present in the SEE. The predictions can also be compared with, for example, a baseline or control state of the PUM traversing such an area, where if the variations are significant, for example exceeding one or more threshold, metric, vector or other dimension, an alert or event may be communicated to the one or more monitoring systems, which can then communicate with the PUM and/or a response system to initiate an appropriate response.

In some embodiments the movements of the PUM as measured by the one or more sensors, devices and/or systems present in the SEE can be used as training data for one or more AI/ML models. This training data may include further data generated by one or more PPE and/or can include one or more sets of context data, for example that generated by the mapping of the environment. The mapping of the environment can include the use of active and/or passive sensors to determine the boundaries of the environment and the positioning of any furniture, furnishing and/or objects within the environment. In some embodiments the environment and the contents thereof, may have one or more risk metrics assigned to each of contents, which can be used, at least in part, to calculate potential impact on a PUM should they interact with them, especially in an adverse manner. For example, falling onto a hard surface can have a higher risk metric than a soft surface.

One aspect of this approach is determining the reach of the limbs of the PUM as they relate to the surfaces of the environment and/or the objects therein, and the potential contact areas thereof. This can include, for example, evaluation, estimation, prediction and/or calculation of the force of those movements, using for example a PPE, such as for example when walking, reaching for an object and the like. These force calculations can, for example, form part of a data set of an AI/ML model both for training and/or prediction. These force calculations can inform the monitoring systems as to the likely or actual consequences of the PUM having contact with the environment, furniture, furnishings and/or objects therein, such that the response systems may be calibrated with a response that is in alignment with such interaction.

In some embodiments, a PPE can include one or more AI/ML models that is configured to evaluate the force exerted by a PUM when making one or more movements. This can, for example, be represented as a force vector, including vector bundles, where for example the force expended by a PUM when making a movement forms part of a multi-dimensional representation of that movement. For example, this can include evaluation and/or prediction of the muscle strength and deployment of those muscles during one or more movements. For example, the force exerted when walking may be represented in three Euclidean dimensions which can form at least two vectors. This measurement can then be used, for example, by the one or more sensors, devices and/or systems to evaluate that movement, for example using a haptic sensor, such that any variation, for example that is not within a set if thresholds, for example those of a quiescent behavior, are identified. These variations can, for example, correspond to difficulties that a PUM is having with their movements, which can be communicated to the monitoring systems.

One aspect is to establish initial resting and/or start state of the position of the PUM, which can include baseline and/or control states, such as one or more quiescent states. For example, this can include determining the overall quiescent state of the PUM represented for example, by their behaviors and the state of the environment represented by the one or more sensors, devices and/or systems of the SEE. For example, a quiescent state can be a PUM sleeping, sitting, reading, eating, walking or other activity where that activity measures within any thresholds determine by the systems measuring the PUM behaviors and/or has any metrics representing risks and safety issues that are also within any thresholds for those activities. In some embodiments the sensors, devices and/or systems of the SEE may be used by one or more PPE to establish a set of reference positions for a PUM, for example standing, sitting, lying and one or more movements, for example reaching, holding objects, grabbing, bending and the like. These reference positions may then be used by the PPE to, at least in part, establish the range of possible movements of the PUM.

In some embodiments, a PPE can be calibrated through measurement and observation of the movements of a PUM as they undertake their daily activities and/or though requesting the PUM to undertake specific movements that can be measured.

In some embodiments there may be multiple models deployed, where for example each model has been generated by one or more AI/ML models. For example, a model may be, at least in part, personalized to a specific PUM, SEE, stakeholder and/or combination thereof.

For example, multi agent systems can be employed in any arrangement, including specialized modules, tools and/or systems which can be calibrated and/or configured for a specific SEE and/or stakeholder, including a PUM. This can, in some embodiments, include one or more actuators, communication systems and/or other sensors, devices and/or systems present in a SEE.

In one or more embodiments, three or more sensors, including for example cameras, may be deployed, for example, in an X, Y, Z axis configuration to capture, at least in part, the movements of a stakeholder, for example a PUM. These movements can be directed and/or undirected, in that the PUM may undertake their daily activities and/or be requested to undertake a specific one or more movements The data sets from these one or more sensors, devices and/or systems employed to monitor the X, Y, Z Euclidean space that include the PUM can be provided to one or more AI/ML models for evaluation. This AI/ML model can then generate predictive outcomes as to these movements, for example, on a joint, limb and/or other body part, including the whole body. These generated outcomes can then form a second data set that can be evaluated by further AI/ML models to establish the physical capabilities of the monitored person. This can include those of a physics engine configured with the laws of physics and/or with personalized physics capabilities of the person being monitored, for example the PUM. In this manner the range of motions and/or force that the monitored person can undertake is used to inform the outcomes of the one or more AI/ML models to ensure that any predicted movements are aligned with the actual capabilities of that person.

For example, in some embodiments, the PUM may be requested to enter an environment that includes multiple sensors, devices and/or systems, for example one or more cameras, lidar, radar and/or other active or passive sensing capabilities, including those can be attached to, worn and/or carried by the person, where for example they are requested to undertake a series of individual and/or sequence of movements, such as walking, sitting, carrying, grabbing, lifting and the like. This can include the person being asked to be at rest in various positions, such that any movement artifacts, for example unconscious movements of the hands, eyes, feet or any other body part can be identified and in some embodiments, may be used to, at least in part, configure a movement artifact PPE to, in part or in whole, filter out such movements from data sets that are presented to other systems, such as further AI/ML models and/or PPE in any arrangement. In some embodiments fixed and/or mobile 3D scanners can be employed as part of the movement measurement, calibration and/or configuration processes.

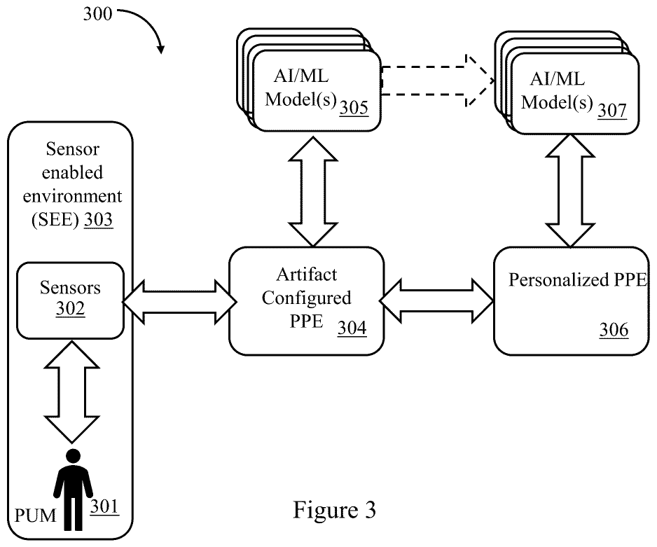

FIG. 3 illustrates an example of a system 300 implementing a configured personalized physics engine 304, according to example embodiments of the present disclosure. A PUM 301 present in a SEE 303, where they are monitored by one or more sensors, devices and/or systems 302 that can generate data sets that are communicated to an artifact configured PPE 304, which can employ one or more AI/ML model 305 for the identification and/or evaluation of movements of the PUM. The artifact configured PPE 304 may generate data sets that are communicated to a personalized PPE 306, which can employ one or more AI/ML models 307. These data sets from the artifact configured PPE 304 may be passed through by the personalized PPE 306 to one or more first AI/ML model 305, or one or more second AI/ML models 307.

Some of these generated data sets may be represented as vectors, which can include metrics for force, acceleration, velocity, time and/or other attributes that can be represented, for example, as higher dimensional spaces, such as Hilbert spaces and the like.

In some embodiments one or more PPE may be configured to represent the interaction of the PUM with a contact surface. For example, if the surface is soft, such as a bed or sofa, the PPE using data sets that present that surface, can emulate the relationship between a PUM movement and that surface. This can include surfaces that, for example, have a degree of risk, such as hotplates, stoves or other heated, cold, wet or otherwise surfaces that have higher risk metrics.

In some embodiments, one or more sensors, devices and/or systems can generate data sets that can be in the form of tokens. These tokens can, for example, represent sets of data which can include for example, movements of one or more stakeholders in a SEE. This can include, for example, sets of data that in aggregate represent patterns, such as those exhibited by a stakeholder when, for example, they undertake a movement or set of movements, such as for example standing, sitting, walking, bending over and the like.

These movement patterns can, in some embodiments, be represented in a PPE, such that the physics of the environment and the movements of the person therein are compliant with the physics of those activities. For example, this can include force of a movement, in that when a stakeholder, for example the PUM, walks the force of their footfall is within a range that is typical for someone of their age, weight, height and/or mobility capability and/or is within previously measured and identified thresholds of that person in such a SEE. In this manner should a person's footfall exceed such a range, an alert may be generated that is communicated to a monitoring system, which can then invoke one or more response systems.

These movements, represented as tokens and, for example, validated by a PPE, can form a set of tokens that can be communicated to an AI/ML models configured to do so, through for example the use of abstract embedding, where, for example, the embedding is in the form of a framework that, for example is generated by a PPE as a representation of a set of movements, expressed as tokens that form, for example, a behavior.

The use of a PPE to provide, for example a framework for the behaviors of a stakeholder, for example a PUM, can be in the form of a set of frameworks for those behaviors, where for example, the typical set of behaviors of a stakeholder in a SEE is represented by the PPE. For example, this can include typical movement arrangements of the stakeholder, for example a PUM, such as sitting, lying, standing, bending over, leaning, reaching in various directions, carrying, pushing, pulling, walking and the like.

Each of these movement arrangements can, in some embodiments comprise a set of tokens, which in part or in whole represent the data sets generated by the one or more sensors, devices and/or systems of the SEE as the stakeholder, for example a PUM, undertakes their daily activities, which include these movement arrangements.

In some embodiments these movement arrangements may be represented as frameworks, where the basic muscular/skeletal movements are represented in the PPE and as the stakeholder is observed in the SEE, these frameworks populated by these data sets such that the PPE becomes a personalized representation of the stakeholder under monitoring. Such an approach, involving calibration and configuration of the PPE, can yield a representation of the movements of a stakeholder, which can be used by, for example an AI/ML model, including for example in one or more digital twins.

In some embodiments a PPE may be configured, including for example using an ML that is connected to that PPE in any arrangement, to represent a health, wellness, care and/or safety event, such as for example a hip replacement, knee or other limb injury and/or corrective action, such as knee surgery or replacement and/or any other procedure and/or event that has, in whole or in part, an impact on the movement capabilities of the PUM. This can include the configuration of the PPE and/or the personalized monitoring systems, including care hubs and/or care processing systems, with data sets that can be representative of the movement and/or other health, wellness, care and/or safety impact of the procedure and/or event, which can include the use of an ML including those configured for movement evaluation and/or prediction, such that the personalized monitoring systems, including the PPE can evaluate, in whole or in part, the movements of the PUM, which can then be used by such monitoring systems to provide communications to, for example, carer, other stakeholders, PUM and/or any other designated and/or authorized person and/or entity, including one or more response systems configured to initiate and/or undertake a response that is aligned with the movement detections.

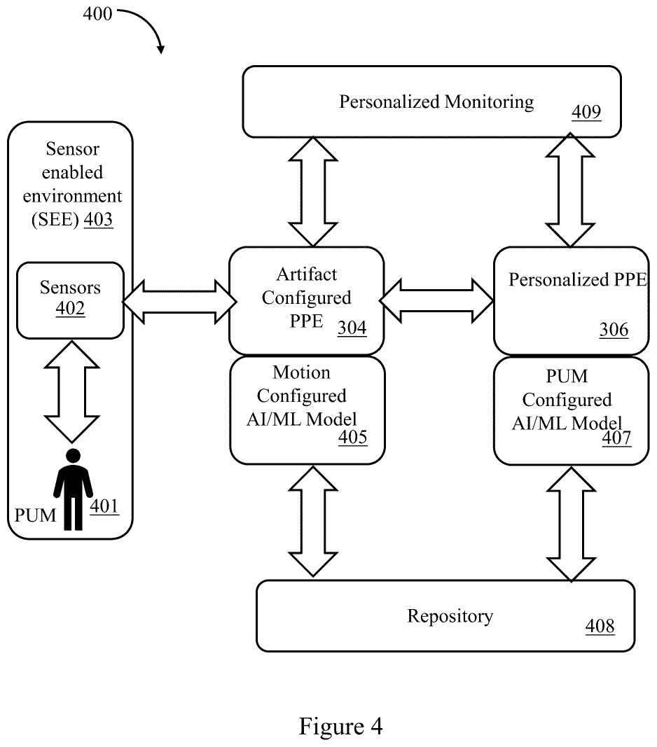

FIG. 4 illustrates an example system 400 providing personalized monitoring 409, according to example embodiments of the present disclosure. A PUM 401 is present in a SEE 403 which includes one or more sensors, devices and/or systems 402 for monitoring the PUM and/or SEE. The data sets generated by the sensors 402 can be communicated to an artifact configured PPE 404, which can employ one or more AI/ML model, including motion AI/ML model 405. The artifact PPE 404 can generate data sets that can be communicated to a personalized PPE 406, which can employ one more AI/ML models 407 configured for a specific PUM 401, such that the data generated by artifact configured PPE 404, motion configured and/or other AI/ML model 405, personalized PPE 406, PUM configured AI/ML model 407 can be communicated to one or more repository 408 and/or one or more personalized monitoring systems 409 in any arrangement.

### Models

The one or more sensors, devices and/or systems present in a SEE can be configured and calibrated to generate data sets that include one or more data value sets, that is the measurements of the sensors, devices and/or systems generate that may be constant or have minimal variations, such that they represent activity in a SEE that is quiescent in nature. In such circumstances where, for example, a sensor is generating data sets, where such data sets are, for example, of a periodic frequency and such sensor is configured, for example including one or more threshold, and as such can detect any changes in state, where for example one or more data sets exceeds the one or more configured thresholds, such data that does not exceed the one or more thresholds can be represented as a count of the individual measurements forming such a data set, for example, a set of values where for example the deviation is insufficient to trigger a state change, and/or an aggregation of the measurements and/or other techniques.

In the situation where such sensors, devices and/or systems are monitoring the movements of one or more stakeholders, including the PUM, similar principles can be applied. For example, the PUM movements may exceed the one or more configuration thresholds of the one or more sensors, devices and/or systems of the SEE and yet may not represent a change of state. For example, even at rest, in for example a quiescent state, a person can exhibit one or more movement artifacts, such as the rise and fall of the chest through breathing, movements of hands and fingers to the face and/or other human movement attributes, habits or minor afflictions.

In some embodiments one or more PPE can be employed to represent these movement artifacts, as they are a part of the representation of the PUM and as such form part of their physical persona. For example, if a person, such as a PUM, touches their face with their hand on a regular basis, the PPE may represent this behavior, involving the movement detections represented by the data sets of the one or more sensors, devices and/or systems present in the SEE, where such data sets represent a pattern, such as face touching, hand movements, shaking or other movement artifacts, such that these are represented as patterns forming, at least in part a quiescent state such that the manner of these occurrences of such activities, including lack thereof, can form, in whole or in part, a behavior of the person in such a quiescent state. These movement artifacts can, in some embodiments be labels as such and can form part of the training data sets of one or more AI/ML models, including those used by a PPE.

In some embodiments there can be multiple PPE employed, for example in a hierarchy or other arrangement. For example, there could be a PPE for artifacts, specialist PPE for body elements, for example limbs and/or body components, for example a hip. There can also be PPE that represent the one or more body elements or systems, such as for example the heart and circulatory system, where for example specialist monitoring systems can generate data sets for such PPE, which can be instantiated in one or more digital twins.

The monitoring and/or evaluation of these potentially inconsequential movement artifacts, as represented by the data sets of the one or more sensors, devices and/or systems of the SEE, could present a situation where large computing resources are required, including those of an AI/ML model to evaluate and consider each individual movement artifact. In some embodiments, the PPE may be configured to represent a set of observed and repeated movement artifacts which are labels as such, classified as typical and that, for example conform to a set of geometric reasoning based on the movements of a set of limbs and/or joints, and match typical human movement behaviors, including those observed by the one or more sensors, devices and/or systems of the PUM. These representations can include the use of manifolds to represent such artifacts. These artifact manifolds can be used to evaluate further movements of the PUM, including those represented by the PPE, to identify those movements that represent intentional movement of the PUM, and consequently represent a change of state from quiescent to another active state.

In some embodiments a PPE instance may be configured to represent these movement artifacts, where for example, this PPE is configured to represent such artifacts and then can operate an adjunct, part of, in collaboration with or in any arrangement for part of a further PPE representing the PUM, including one or more AI/ML models. For example this can include the use of self-attention and contextual vectors, where for example, data sets form the one or more sensors, devices and/or systems present in a SEE can be arranged and evaluated to in part or in whole remove any movement artifacts and/or other noise form a stream of data, This can include each of the one or more sensors, devices and/or systems undertaking initial processing of the measurements of the SEE, where for example, the calibration and/or configuration of the one or more sensors, devices and/or systems includes, for example one or more thresholds and/or data values. In some embodiments, such determinations may include the use of one or more game theory games to, at least in part, determine whether certain data should be communicated to one more other sensors, devices and/or systems.

This configuration of the PPE could then, representing those “unconscious” movement artifacts, be employed as a filter on the observed movement artifacts of the monitored person, including the PUM. In this manner those artifacts can form part of the monitoring, where for example counts or other metrics of these artifacts may be used to, at least in part, detect any changes in the state, frequency, occurrence, timing or other attributes of these artifacts.

In some embodiments, patterns and/or sequences of these artifacts may be observed and represented in a PPE, where for example they may have relationships with one or more patterns and/or behaviors of the PUM. For example, an artifact, sequence of and or pattern of artifacts may occur prior to or after a behavior that, for example, has a potential health, wellness and/or safety consequence, and as such may form part of a set of patterns or behaviors. In this example, a comparison of a PPE configured to represent the artifact or set thereof, may form part of the monitoring operations, using minimal monitoring resources, and when the behavior with potential consequence is initiated, monitoring resources, form example, sensors, devices and/or systems present in the SEE, may be configured and directed at that behavior.

These artifacts and behavior arrangements may form part of a quiescent state of the monitoring of the PUM, in that any artifacts are within the boundaries, thresholds and/or other metrics of that state, however any variations of these artifacts can indicate a change in behavior and/or variation that exceeds one or more thresholds, potentially indicating an event and/or state change, the identification and detection of which can provide indicators for potential wellness, health, safety and/or care issues at the earliest possible opportunity.

In some embodiments, separation of movements such as incidental movement artifacts, from activities representing behaviors can be undertaken by employing one or more PPE and/or other AI/ML model, can include, for example, grouping non-active behavior instigating movements into patterns, which although representative of a stakeholder's individual movement artifacts, do not lead to behaviors that have intent. For example, fiddling with the fingers of an object, for example a pen, or brushing the face, clasping the hands and the like, may all be “tells” as to the PUM's intentions or concerns, however they may or may not precede an intentional behavior, such as walking to the kitchen, picking up a book or TV remote and the like.

These artifacts can, in some embodiments, be evaluated as noise of the signal of the intentional behaviors, however one or more monitoring systems, including care hubs and/or care processing systems may be used to recognize these artifacts and, in some embodiments, provide such recognition to one or more AI/ML models to, for example enhance and/or extend their models and/or representations of the PUM and their activities.

In some embodiments, movement artifacts can form part of a context, for example they may be represented in a movement language, for example represented as tokens. For example, such an approach can be employed as part of a previously trained movement artifact model used by one or more PPE, including specialist PPE, that can, for example be deployed with the one or more sensors, devices and/or systems of the SEE, forming the edge sensing for such SEE and/or as a RAG and/or Filter for one or more LLM, LVM, LCM, convolutional neural network, and/or other AI/ML models in any arrangement.

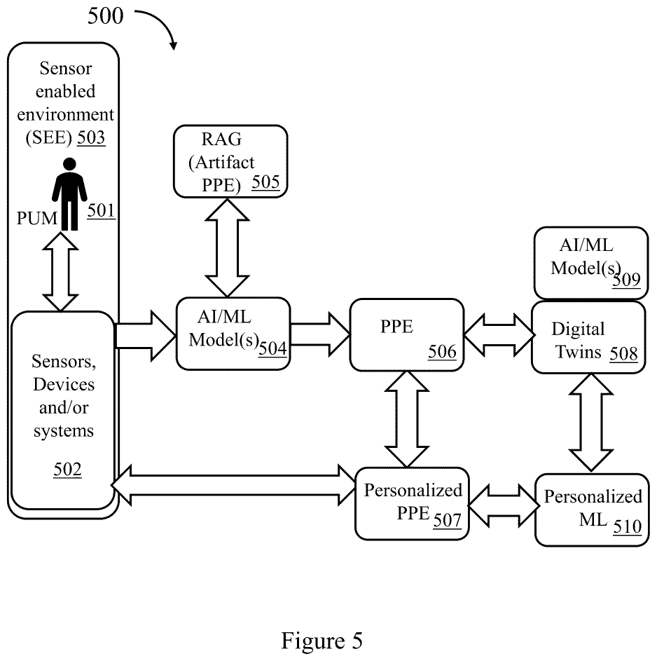

FIG. 5 illustrates an example system 500 incorporating digital twins 508, according to example embodiments of the present disclosure. A PUM 501 is present in a SEE 503 which includes one or more sensors, devices and/or systems 502 which can monitor the movements and/or behaviors of the PUM 501. The data sets generated by the sensors 502 can be communicated to one or more AI/ML models 504, including motion configured AI/ML model. These AI/ML models can communicate their output to one or more PPE 506, which can communicate with one or more digital twins 508 that can employ one or more AI/ML models 509. In some embodiments such data sets from sensors 502 may be contemporaneously communicated to a personalized PPE 507 configured for the PUM 501 present in the SEE 503 and the data generated by the PPE 507 can be communicated to one or more PPE 506 and/or a personalized AI/ML model 510 that employes a model of the PUM 501 and the SEE 503.

This can include the personalization of such models, including training models and can, in some embodiments, be used to establish correlations and/or causations of events and/or actions of a PUM or other stakeholder which have risk attributes. In some embodiments, such movements can be represented as a direct “copy” of actual for patterns and/or behaviors represented by models, where for example, using one or more digital twin, PPE, and/or AI/ML models, of these movements can from a comprehensive representation of a PUM in a SEE.

In some embodiments, simulated environments can be employed to develop, at least in part, game-based training data which can be used by an AI/ML model as part of the development of a model that represents the environment of the SEE. This can include identification of contact areas that a PUM or other stakeholder is likely to interact with, either on an intentional basis, such as a floor, or on a risk adjusted basis, such as a wall, furniture or other impediments.

In one or more embodiments, the present disclosure also relates to multi-stage monitoring architecture to improve the functioning of sensor-based monitoring systems. For example, the multi-stage monitoring architecture may enforce personalized physics constraints that reject non-physically plausible joint motions and surface interactions prior to inference. Additionally, the multi-stage monitoring architecture may filter movement artifacts (e.g., tremor and fidget bands between 4-12 Hz) before baseline updates to reduce drift and bandwidth. Further, the multi-stage monitoring architecture may adapt detection thresholds from quiescent baseline intervals and an energy-budget estimate. The multi-stage monitoring architecture may also actively reconfigure sensors (activation, sampling rate, measurement granularity) based on predicted contact areas from an environment map. Representative implementations from the multi-stage monitoring architecture achieve lower false-alert rates and reduced end-to-end latency compared to models lacking the physics constraints, artifact filtering, and sensor-control operations.

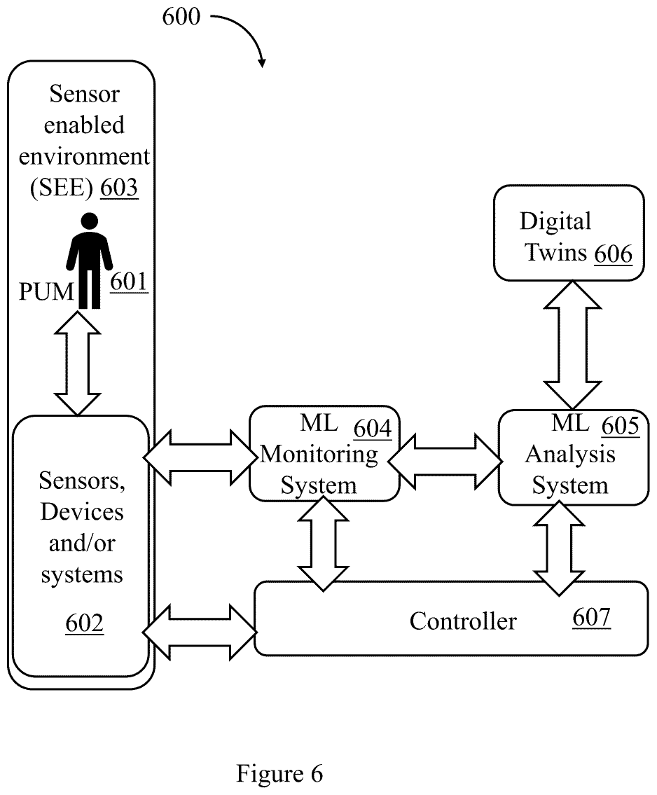

FIG. 6 illustrates an example system 600 incorporating an ML monitoring system 604, ML analysis system 605, according to example embodiments of the present disclosure. A PUM 601 is present in a SEE 603 which includes one or more sensors, devices and/or systems 602 which can monitor the movements and/or behaviors of the PUM 601. The data sets generated by the sensors 602 can be communicated to the ML monitoring system 604, including motion configured ML. The ML monitoring system 604 communicates their output to the ML analysis system 605, which can communicate with one or more digital twins 606 that can employ one or more MLs. Both the ML monitoring system 604 and the ML analysis system 605 may be one or more AI/ML models.

In one or more embodiments, the one or more sensors, devices and/or systems 602 may include motion sensors, mm Wave radars, cameras, WiFi-based location sensors, wearable accelerometers, gyroscopes, altimeters, microphones, pressure mats, radar-Doppler sensors, capacitive sensors, 3D scanners, inertial measurement units (IMUs), or biometric sensors, in any combination. These sensors 602 may be worn, carried, embedded in the environment, mounted in fixed positions, or implemented as distributed sensing modules throughout the SEE 603. The sensors 602 may continuously generate multimodal measurements including movement, posture, environmental interaction, force, acceleration, and other physical or contextual attributes associated with the PUM 601.

The ML monitoring system 604 is configured to receive the multimodal data from the sensors 602 and perform initial movement detection and extraction of a set of values of movement parameters. In some embodiments, the ML monitoring system 604 includes motion-configured ML models, feature-extraction modules, sequence detectors, and/or preprocessing engines configured to identify a movement type exhibited by the PUM 601. Additionally, the ML monitoring system 604 can determine the set of values of movement parameters values, including joint angles, movement vectors, body orientation, limb trajectories, spatial location, temporal duration, force indicators, or other kinematic attributes. Furthermore, the ML monitoring system 604 may further convert sensor inputs into movement representations which include machine-interpretable tokens or vectors encoding the identified movement type and corresponding parameter values, thus generating structured outputs suitable for downstream physics-based or ML-based analysis.

Still referring to FIG. 6, the ML analysis system 605 is configured to receive the movement representations from the ML monitoring system 604 and perform higher-level evaluation, classification, and risk assessment. The ML analysis system 605 may include additional machine-learning models, such as transformer-based motion evaluators, temporal-sequence analysis engines, pattern-recognition modules, or personalized mobility-assessment models. Based at least in part on the movement representations, the ML analysis system 605 may identify and report changes in a condition of the PUM 601. Based on the condition of the PUM 601, the ML analysis system 605 outputs a risk metric related to the PUM's mobility, stability, fatigue, or movement consistency. It is further envisioned that the ML analysis system 605 may compute a deviation metric relative to a baseline movement profile of the PUM and trigger an alert when the deviation exceeds an adaptive threshold. Such analysis may incorporate high-dimensional embeddings, contextual metadata, physics-based joint-angle validity, and temporal-behavioral correlations.

In one or more embodiments, the ML analysis system 605 may be in communication with one or more digital twins 606 representing a model of the human musclo-skeletal model (i.e., a Musculo-skeletal computational model). The digital twins 606 may be customized to produce a personalized biomechanical, behavioral, or musculoskeletal computational model of the PUM 601. For example, the digital twin 606 may store historical movement data, baseline behavioral profiles, anatomical constraints, and personalized joint-range limits. The ML analysis system 605 may update the digital twin 606 using validated movement data from the ML monitoring system 604 and may query the digital twin 606 to determine whether observed movements are physically plausible, within the PUM's known capabilities, or indicative of deterioration or risk. The digital twin 606 may therefore operate as a personalized reference model against which the ML analysis system 605 detects deviations or abnormal movement patterns.

In some embodiments, the system 600 may include a controller 607 in communication with the sensors 602 and configured to reconfigure one or more sensors based on the movement type identified by the ML monitoring system 604 or the condition or risk determined by the ML analysis system 605. Sensor reconfiguration may include, for example, activating or deactivating sensors, adjusting sampling rates, modifying measurement granularity, selecting between high-resolution or low-power modes, or directing particular sensors to capture enhanced detail in response to detected movement anomalies. Through such dynamic sensor-management operations, the system 600 may optimize power usage, reduce noise, increase accuracy of movement capture, or improve timeliness of emergency detection.

In one or more embodiments, the system 600 may also include security protocols for communication. For example, the sensors 602, ML monitoring system 604, ML analysis system 605, and digital twin 606 may communicate using authenticated channels or secure tokens containing movement-analysis payloads, metadata, and access-control attributes. In some embodiments, the system 600 may operate in a multi-stage pipeline, where token-generation, physics-based validation, and higher-level inference are performed in a staged or parallel manner. The system 600 may output alerts, risk metrics, and movement-analysis results to downstream care, wellness, and safety processing systems, remote monitoring hubs, automated response systems, or caregiver endpoints.

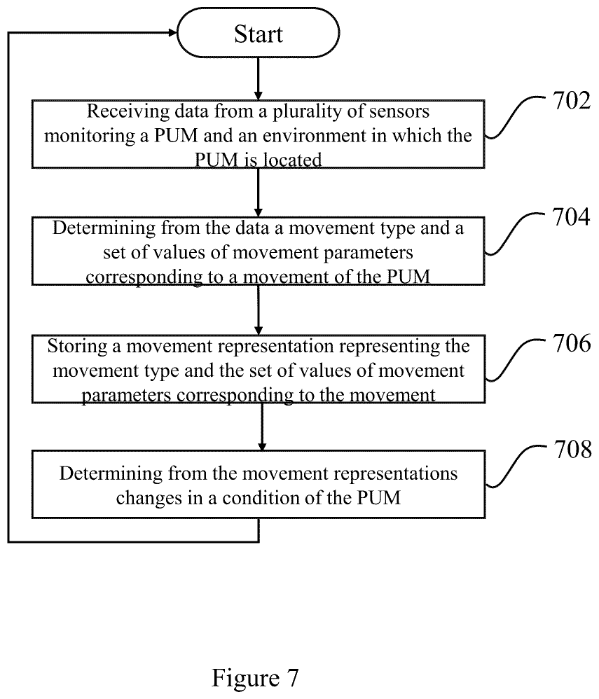

FIG. 7 shows an example flowchart in accordance with one or more embodiments of a general method for monitoring movements of the PUM. While the various steps in FIG. 6 are presented and described sequentially, one of ordinary skill in the art will appreciate that some or all the steps may be executed in different orders, may be combined or omitted, and some or all the blocks may be executed in parallel. Furthermore, the steps may be performed actively or passively.

In Step 702, the system receives data from a plurality of sensors monitoring a PUM and an environment in which the PUM is located. The data includes multimodal, time-stamped measurements characterizing movement and context of the PUM. The plurality of sensors may include worn, carried, or embedded sensors, and environmental sensors such as accelerometers, gyroscopes, altimeters, mmWave radars, cameras, LiDARs, microphones, ultrasonic sensors, and pressure sensors. These sensors continuously collect timestamped data on a movement representation. The plurality of sensors may further include preprocessing modules that format or quantize the raw incoming measurements before they are used in downstream processing. The preprocessing may include formatting, quantization, denoising, windowing, and optional fusion to produce a movement-measurement set suitable for subsequent movement-type determination and parameter extraction.

In Step 704, the data from the sensors is used to determine a movement type and a set of values of movement parameters corresponding to a movement of the PUM. For example, The data record from the sensors is encoded with a detected movement including at least the movement type and the set of values of movement parameters. The resultant structured movement record may encode, by way of example, limb motion, body posture, joint rotation, environmental interactions, spatial position, timing/duration, velocity/acceleration, and force-vector components, as applicable to the detected movement. It is further envisioned that one or more movement-analysis AI/ML models (e.g., neural networks or transformer-based models) execute feature extraction and classification functions to output the movement type and the associated parameter values in a form ready for tokenization.

In Step 706, the system stores a movement representation representing the movement type and the set of values of movement parameters corresponding to the movement. In one or more embodiments, the system generates the movement representation based on the movement type and the set of values movement parameters for storage. For example, one or more AI/ML models (e.g., motion-configured ML systems, LLMs/SLMs, CNNs, RNNs, graph-based encoders, state-space models, or transformers) convert incoming measurement streams into the machine-interpretable tokenized movement representations that capture movement modules, movement frameworks, patterns, behaviors, and complex joint/limb sequences, and optionally augmented with contextual metadata (e.g., time-of-day, location, intent indicators). In some embodiments, the movement representations may be subjected to physics-based validity evaluation (e.g., verifying joint-angle limits and feasible musculoskeletal trajectories) to ensure that non-physical data does not propagate to later stages. The physics-based validity may also include verifying that observed joint angles do not exceed anatomical limits and that limb trajectories conform to feasible musculo-skeletal constraints.

In Step 708, from the movement representations, changes in a condition of the PUM are determined. Based on the changes in the condition of the PUM, a risk metric is output indicative of the assessed condition. The condition of the PUM may encompass mobility, balance, fatigue level, movement success metrics, and/or deviation from the PUM's typical movement capability, among other factors. The risk metric may indicate or signal, for example, a potential fall, a deterioration in mobility, an increased risk condition, a significant change from baseline behavior, or an emergency that warrants human and/or automated response. It is further envisioned that risk outputs may be consumed by caregiving systems, monitoring hubs, or response modules to initiate mitigations.

In some embodiments, a deviation metric is computed between the movement representations and a baseline movement profile of the PUM. The deviation metric triggers an alert when the deviation metric exceeds an adaptive threshold. The deviation metric may be expressed, for example, in terms of high-dimensional vector distances, topological differences, temporal divergence, and/or physics-based mismatches between predicted and observed sequences. The baseline movement profile may be derived from quiescent-state intervals reflecting low-variance routine activity for the PUM. The adaptive threshold may adjust dynamically (e.g., based on recent variance or energy considerations) to reduce false positives while enabling rapid detection of emergent conditions such as weakness, imbalance, tremor progression, fatigue, reduced range of motion, or loss of mobility. Additionally, deviation metrics may reflect emergent conditions such as weakness, imbalance, tremor progression, fatigue, reduced range of motion, or loss of mobility. Alerts may be communicated to caregivers, remote monitoring systems, or automated response modules, or optionally accompanied by recommended mitigations.

In some embodiments, the baseline movement profile may be maintained and updated over time using validated, representative windows of movement data, thereby preserving a stable yet adaptive reference that tracks gradual mobility changes while resisting short-term noise. The adaptive threshold may be computed using statistics of quiescent intervals and/or trend analyses across trailing windows, so that sensitivity aligns with the PUM's current variability and condition. In some implementations, when no alert condition is met, the system updates the baseline with artifact-free, representative measurements, whereas windows deemed non-representative or non-physical are down-weighted or excluded from baseline updates.

In one or more embodiments, in response to any of the Steps 702-708, one or more sensors of the plurality of sensors may be reconfigured. Such reconfiguration may include activating or deactivating sensors, increasing or decreasing sampling rates, and/or changing data granularity or spatial/temporal resolution, to reduce bandwidth and power during low-risk intervals and increase fidelity during higher-risk or diagnostically relevant intervals.

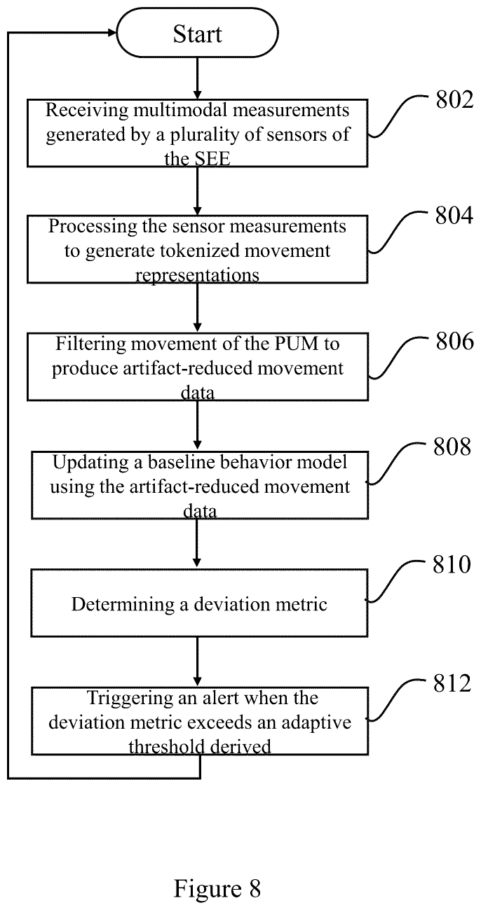

FIG. 8 shows an example flowchart in accordance with one or more embodiments of a general method for monitoring movements of the PUM using SEE, a first-stage AI/ML model, PPE, and a digital twin. While the various steps in FIG. 8 are presented and described sequentially, one of ordinary skill in the art will appreciate that some or all the steps may be executed in different orders, may be combined or omitted, and some or all the blocks may be executed in parallel. Furthermore, the steps may be performed actively or passively.

In Step 802, the system receives multimodal measurements generated by a plurality of sensors of the SEE. For example, the SEE may include worn, carried, or embedded sensors, and environmental sensors such as accelerometers, gyroscopes, altimeters, mmWave radars, cameras, LiDARs, microphones, ultrasonic sensors, and pressure sensors. These sensors continuously collect timestamped data on a movement representation. The movement representation is a data record that encodes a detected movement including at least movement type and values of one or more movement parameters. For example, the movement representation may be limb motion, body posture, joint rotation, environmental interactions, spatial position, and ambient conditions. The SEE may further include preprocessing modules that format or quantize the raw incoming measurements before they are used in downstream processing.

In Step 804, a first-stage AI/ML model processes the sensor measurements to generate tokenized movement representations. The first-stage AI/ML model may include motion-configured ML systems, LLMs, SLMs, CNNs, RNNs, graph-based encoders, state-space models, or transformer architectures. The first-stage AI/ML model may be configured to analyze changes in joint positions, limb orientations, and temporal movement sequences. The first-stage AI/ML model converts the incoming multimodal sensor streams into structured representations, which may take the form of tokens, embeddings, vectors, or other quantized data structures suitable for downstream physics-based evaluation. For example, these tokenized representations may capture movement modules, movement frameworks, patterns, behaviors, or complex sequences of joint and limb interactions, and may incorporate contextual data such as time, location, and intent indicators. It is further envisioned that the physics-based evaluation may be a physics-based validity that includes verifying that observed joint angles do not exceed anatomical limits and that limb trajectories conform to feasible musculo-skeletal constraints.

In Step 806, the PPE filters movements of the PUM to produce artifact-reduced movement data. For example, the PPE evaluates the tokenized movement representations to remove movement artifacts and apply physics-based constraints. It is further envisioned that removing movement artifacts may include tremor-band components between 4-12 Hz prior to updating the baseline behavior model. The PPE uses a personalized musculo-skeletal computational model of the PUM to determine whether proposed movements are physically plausible. The PPE may enforce joint-angle limits, geometrical constraints, velocity and acceleration bounds, force-vector restrictions, and surface-interaction models (e.g., soft surfaces, hard surfaces, hot or slippery surfaces). The PPE also identifies and filters movement artifacts such as tremors, fidgeting, unconscious motions, breathing-induced subtle movements, or measurement noise. In some embodiments, the PPE operates as a gatekeeper or “filter stage” that prevents erroneous or non-physical data from contaminating digital-twin baselines.

In Step 808, a digital twin of the PUM is updated using the artifact-reduced movement data output by the PPE. As taught in the specification, the digital twin represents a personalized biomechanical and behavioral model of the PUM, including individualized limb ranges, motion patterns, movement capabilities, and profile-specific constraints. The cleaned movement data is used to update the baseline behavior model, which reflects the PUM's typical or quiescent movement patterns. The system may exclude data segments identified as artifacts or non-representative. Baseline updates may occur continuously or within predefined windows, allowing the digital twin to track gradual changes in mobility, gait, energy expenditure, or physical condition. It is further envisioned that updating the digital twin includes rejecting, by the PPE, movement data that violates joint-angle limits or contact-surface constraints, thereby reducing false event detection and computational load.

In Step 810, the system computes a deviation metric that measures how current movement behavior compares to the stored baseline behavior of the digital twin. The deviation may be expressed using high-dimensional vector distances, topological differences, temporal divergence, or physics-based mismatches between predicted and observed movement sequences. Deviations may reflect emergent conditions such as weakness, imbalance, tremor progression, fatigue, reduced range of motion, or loss of mobility. Computed deviation metrics may incorporate manifold-based representations, contextual vectors, embeddings, or multi-modal fused features generated earlier in the AI/ML model.

In some embodiments, the deviation metric is compared against an adaptive threshold determined from quiescent-state behavioral baselines and estimated energy budgets of the PUM. The specification explains that quiescent states are representative resting or routine-activity patterns of the PUM, providing stable reference frames for low-variance movement. In some embodiments, the system dynamically adjusts thresholds based on recent energy expenditure, measured fatigue, circadian factors, or movement success metrics. Adjustments may be computed using exponential moving averages, weighted context vectors, trend analyses, or game-theoretic evaluations of PUM state. The adaptive threshold ensures that alerts are not triggered by benign variations while enabling rapid detection of significant deviations in physical capability.

In Step 812, the system determines whether the deviation metric exceeds the adaptive threshold. If so, the system identifies a change in the condition of the PUM and triggers an alert. The condition of the PUM may be a physical condition including mobility, balance, fatigue level, movement success metrics, or deviation from baseline movement capability. The alert may indicate a potential fall, a deterioration in mobility, an increased risk condition, a significant change from baseline behavior, or an emergency requiring human or automated response. Alerts may be communicated to caregivers, remote monitoring systems, or automated response modules. The system may also generate suggested mitigations or recommendations, such as increasing sensor sampling rates, adjusting sensing granularity, or activating additional sensors to further evaluate the condition.

In some embodiments, the system may reconfigure one or more sensors of the SEE based on the movement type, risk condition, or predicted contact areas of the PUM. Such reconfiguration may include activating or deactivating sensors, increasing or decreasing sampling rates, changing granularity, adjusting spatial or temporal granularity, realigning sensor orientation, or enabling higher-resolution data capture. This dynamic sensor-control capability allows the system to reduce bandwidth and computation during low-risk intervals and increase resolution and sensitivity during high-risk or diagnostically relevant intervals.

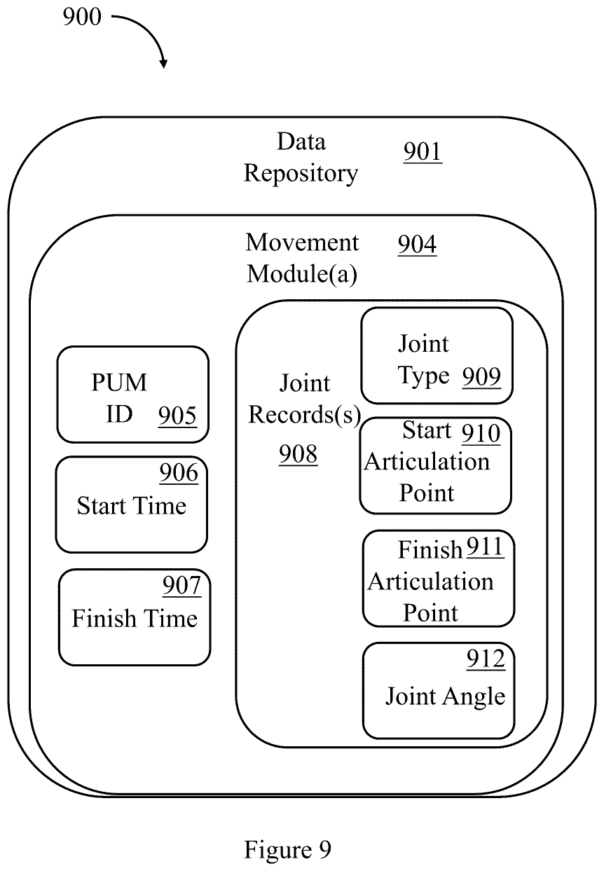

FIG. 9 illustrates an example data structure 900 stored in a data repository 901, according to example embodiments of the present disclosure. The data repository 901 includes data sets that are processed and organized into one or more movement modules 904. Each movement module 904 represents a quantified, elemental unit of movement for at least one joint or body segment of a PUM, or some other object, and may correspond to a discrete, temporally bounded articulation detected by the SEE. The data repository 901 may store these movement modules as individual records or as part of larger sequences, enabling downstream evaluation by one or more AI/ML models, digital twins, and/or PPE.

In some embodiments, the movement module 904 includes a PUM identification (ID) 905 that uniquely associates the movement module 904 to the PUM. The movement module 904 additionally includes a start time 906 and a finish time 907, which together define the temporal window in which the corresponding movement occurred. The movement module 904 may further include one or more joint records 908, each describing a specific joint or articulation measured during the movement. Each joint record 908 may include: a joint type 909 identifying the anatomical joint (e.g., knee, elbow, wrist, hip); a start articulation point 910 expressed as three-dimensional coordinates (X, Y, Z); a finish articulation point 911 expressed as corresponding three-dimensional coordinates (X, Y, Z); and a joint angle 912 representing the rotational or angular displacement of the joint over the duration of the movement. In some embodiments, the joint records 908 may further reflect kinematic, anatomical, or biomechanical constraints enforced by a PPE to ensure that the measured movement is physically plausible and within the anatomical limits of the PUM. Such constraints may be derived from a digital twin of the PUM, thereby improving the accuracy and reliability of the movement module 904.

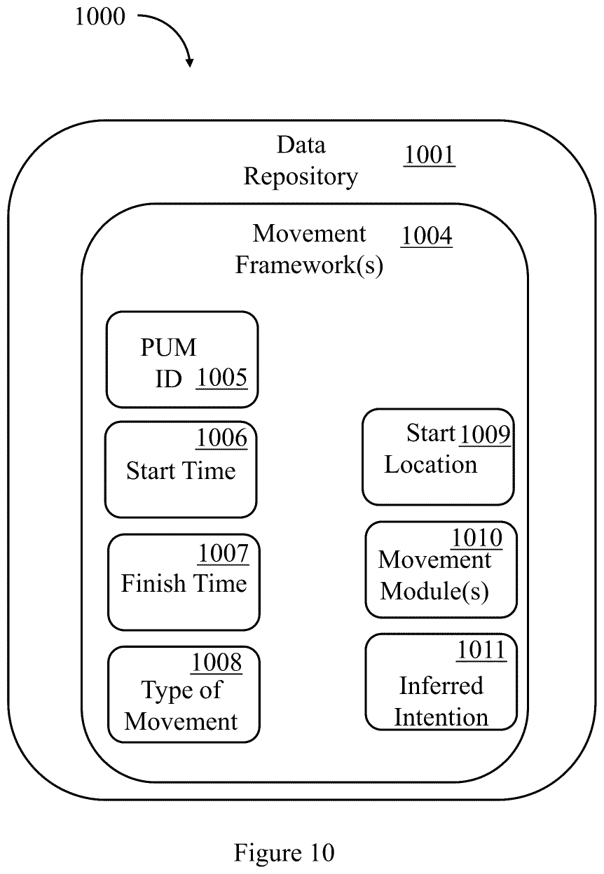

FIG. 10 illustrates an example data structure 1000 stored in data repository 1001, according to example embodiments of the present disclosure. Similar to the movement module, the data repository 1001 includes data sets that are processed to form one or more movement frameworks 1004. Each movement framework 1004 represents a higher-level, aggregated sequence or collection of movement modules that together characterize a more complex movement, activity, or behavior of the PUM. For example, the movement framework 1004 may represent a standing-to-walking transition, a reaching activity, or a series of joint motions forming a recognized pattern or behavior. In some embodiments, the data repository 1001 may maintain the movement frameworks 1004 as structured records accessible by downstream systems for behavior recognition, trend analysis, or risk detection.

In some embodiments, the movement framework 1004 may include a PUM identification (ID) 1005, a start time 1006, and a finish time 1007 defining the temporal interval associated with the overarching movement or activity. The movement framework 1004 may further include a type of movement 1008, which can categorize or classify the movement (e.g., standing up, sitting down, reaching, gait behavior, or other identified patterns). Additionally, the movement framework 1004 may include a start location 1009 that represents the initial spatial position of the PUM at the commencement of the movement sequence. The movement framework 1004 may include one or more movement modules 1010, which serve as the elemental units composing the full activity. In some embodiments, the movement framework 1004 may further include an inferred intention 1011 that reflects a determination, made for example using one or more AI/ML models, of the likely purpose, goal, or intention underlying the activity represented by the sequence of movement modules. This inferred intention 1011 may be used to enhance prediction, monitoring accuracy, behavioral modeling, or risk assessment for the PUM, including early identification of deviations from baseline activity patterns.

### Examples

In one or more illustrative examples, the SEE includes two wrist-mounted inertial measurement units (IMUs) sampling linear acceleration and angular velocity at 100 Hz each, a chest-mounted IMU also sampling at 100 Hz, a floor-embedded pressure mat sampling at 200 Hz, and a 77 GHz mm Wave radar generating range-Doppler frames at 20 Hz to detect limb displacement and coarse body posture. The incoming raw streams are temporally aligned using timestamp interpolation into 50 ms analysis windows, and IMU signals are low-pass filtered at 20 Hz to remove high-frequency noise. Each fused window is represented as a 256-dimensional movement embedding generated by a transformer-based first-stage model trained on movement modules and movement frameworks, as described elsewhere. The embeddings include contextual tags indicating time-of-day and room location. The PPE evaluates each embedding by verifying that inferred limb joint angles lie within the PUM's personalized musculo-skeletal limits, for example rejecting windows indicating knee flexion exceeding approximately 135 degrees or neck rotation exceeding approximately 90 degrees, based on the PUM-specific digital-twin anatomical model. Windows exhibiting tremor-band energy between 4-12 Hz above a learned threshold is marked as movement artifacts and excluded from baseline updates to prevent drift.

In some illustrative examples, the digital twin maintains a rolling seven-day exponential-moving-average (EMA) baseline of the PUM's typical movements. For each validated movement embedding that passes PPE filtering, the digital twin updates its baseline profile using:

$${Baseline}_{t} = α \cdot {Embedding}_{t} + ( 1 - α ) \cdot {Baseline}_{t - 1} , \qquad Equation 1$$

where Baselinet is the updated behavioral or movement baseline at time t, Xt is the current movement measurement or embedding, a is a smoothing coefficient with a=0.10 providing gradual adaptation to long-term mobility changes while resisting short-term noise, typically between 0.05 and 0.2, and Baseline-1 is the prior baseline value. The EMA establishes a stable but adaptive representation of the PUM's typical movements. It smooths short-term variability while enabling detection of gradual long-term changes in mobility or therapy progress.

To detect health- or mobility-related changes, the system computes a Mahalanobis distance between the current embedding vector and the baseline cluster defined by historical embeddings and their covariance:

$$D = {( x - μ )}^{T} {\sum }^{- 1} ( x - μ ) , \qquad Equation 2$$

where D is the deviation metric comparing current movement to baseline, x is the current movement embedding, μ is the baseline mean embedding, Σ is the covariance matrix representing baseline variability, Σ⁻¹ is the inverse covariance matrix, and T denotes vector transpose. T is automatically adapted based on quiescent-state intervals—indicates a drift from the PUM's established movement baseline. For example, the system may maintain T such that the expected false-alert rate remains below one alert per day, increasing or decreasing T dynamically depending on observed variance during quiescent movement periods. When D exceeds T, the system flags a condition change and triggers an alert. This deviation metric quantifies how far a current movement is from baseline behavior in a multi-dimensional embedding space, allowing precise detection of subtle abnormalities. It is particularly effective for identifying early mobility changes that would not appear in single-dimension thresholds.

In some illustrative examples, the system derives a real-time energy-budget estimate for the PUM from IMU-measured step force, torso acceleration, and pressure-mat-derived gait cadence. The system compares these quantities to a personalized metabolic equivalent (MET) model stored in memory and determines a predicted residual daily energy level. When the residual energy falls below a threshold-indicating fatigue—the system automatically reduces the alert threshold used for deviation detection, making the system more sensitive to instability or unsafe movements. In also response to predicted fatigue or rising risk scores, the controller may reconfigure sensors in the SEE by increasing the radar sampling rate from 20 Hz to 40 Hz, enabling high-resolution gait capture, or by activating additional IMU sensors previously in a low-power state. Conversely, during low-risk quiescent intervals, the system may decrease sampling frequency or deactivate redundant sensors to reduce bandwidth and computational load. These dynamic and context-dependent sensing adjustments enable the system to operate with improved efficiency and earlier detection of mobility decline.

In some examples, the PPE determines whether a movement remains within the PUM's anatomically feasible range. To compute this, the PPE may use a geometric method for computing joint angles from limb-segment vectors, the joint-angle kinematics may be computed using the dot product formula:

$$θ = {cos}^{- 1} ( \frac{u * v}{‖ u ‖ ‖ v ‖} ) \qquad Equation 3$$

where θ represents the joint angle formed between two connected limb segments, u and v are limb-segment vectors (e.g., upper arm and forearm), u·v denotes the dot product of the two vectors, and |u| and |v| are magnitudes (lengths) of the limb-segment vectors.

In some implementations, deviations in knee or hip angular velocity profiles from baseline (e.g., slower extension during sit-to-stand) indicate deterioration in functional performance. This can be solved for a given angle using:

$$ω ( t ) = \frac{d θ ( t )}{dt} , α ( t ) = \frac{d ω ( t )}{dt} \qquad Equation 4$$

where ω is the angular velocity, i.e., the rate of change of the joint angle, α is the angular acceleration, i.e., the rate of change of angular velocity, θ is the joint angle, and t is time.

In one or more embodiments, torque quantifies the rotational effort applied at a joint and can indicate excessive strain or compensatory movements, which are clinically relevant in PT and injury-recovery monitoring. The PPE may compare computed torque against personalized musculo-skeletal limits to evaluate safe execution of functional tasks using:

$$τ = r \times F \qquad Equation 5$$

where τ represents torque applied at a joint, r is the vector from the joint center to the point of force application, and F is the applied force vector.

In another example, linear force relationships allow the system to estimate forces generated during limb and whole-body motion from measured accelerations, enabling assessment of effort, stability, and gait quality. In PT applications, reduced or asymmetric force output can indicate deterioration or uneven loading. The PPE evaluates whether resulting forces exceed physical therapy-safe limits or differ from previous baselines (e.g., decreasing push-off force during gait cycles) using:

$$F ( t ) = m \cdot a ( t ) \qquad Equation 6$$

where F is the force produced by or applied to a limb or body segment, m is mass (segment mass or body mass, depending on context), a is linear acceleration measured by inertial sensors, and t is time.

In another example, ground reaction force (GRF) estimation helps characterize gait cycles and balance by measuring vertical loading forces during stance. This supports early detection of weakness, fall-risk patterns, and compensatory gait behaviors. The PPE evaluates GRF such that a reduced GRF during stance can indicate weakness and an elevated GRF irregularities may indicate fall risk or pain-avoidance gait using:

$$GRF ( t ) = m ( g + {a}_{z} ( t ) ) \qquad Equation 7$$

where GRF(t) is the estimated vertical ground reaction force at time t, m is body mass, g is gravitational acceleration, and a_{z}(t) is vertical acceleration from wearable or environmental sensors.

In some examples, the system computes a residual daily energy estimate and lowers detection thresholds when fatigue is predicted based on a kinetic-energy-based expenditure model. This expression models kinetic energy plus mechanical work, allowing estimation of the PUM's energy use across daily activities. When compared against baseline, changes in energy expenditure can indicate fatigue, frailty progression, or physical-therapy improvement. The kinetic-energy-based expenditure model may be used as follows:

$$E ( t ) = \frac{1}{2} m {v ( t )}^{2} + {\int }_{0}^{t} F ( τ ) v ( τ ) d τ \qquad Equation 8$$

where E(t) is estimated energy expenditure over time t, m is mass, v(t) is instantaneous velocity of a limb or body segment, F(τ) is force at prior time τ, and ∫Fv dt represents mechanical work done.

In some examples, the system computes a joint-trajectory smoothness (Rehab Quality Metric) following:

$$Jerk ( t ) = \frac{{d}^{3} x ( t )}{{dt}^{3}} \qquad Equation 9$$

where Jerk(t) is the third derivative of position, indicating sudden motion changes and x(t) is limb or body-segment position over time t. Jerk captures rapid changes in movement, making it sensitive to tremor, instability, and poor motor control. Monitoring jerk helps identify neurological decline or improvements during physical-therapy rehabilitation. A Higher jerk correlates with poor motor control (stroke, Parkinson's, MS).

In one or more examples, the PPE incorporates a gait cadence and step-length estimation. For example, the PPE uses PUM-specific baselines and flags asymmetry based on:

$$cadence = \frac{{N}_{steps}}{Δ t} , L = {\int }_{{t}_{1}}^{{t}_{2}} {v}_{forward} ( t ) dt \qquad Equation 10$$

where Nₛₜₑₚₛ is the number of steps detected, Δt is the time interval over which steps are counted, L is step length, v_{forward}(t) dt is forward velocity during a gait cycle, and t₁, t₂ define a single step interval. Cadence and step length provide fundamental gait parameters for assessing mobility, fatigue, and PT recovery. Deviations from baseline can indicate imbalance, pain-avoidance patterns, or disease progression.

In some examples, the PPE and/or the digital twin can model slow rehabilitative improvements using a ROM progress curve model following:

$${θ}_{max} ( t ) = {θ}_{max} ( 0 ) + k ( 1 - {e}^{- ct} ) \qquad Equation 11$$

where θₘₐₓ(t) is maximum achievable ROM at time t, θₘₐₓ(0) is baseline ROM measured at therapy start, k is a parameter representing total expected improvement, c is the recovery-rate constant, and t is elapsed therapy time. This curve models gradual improvement in joint ROM during rehabilitation, allowing the digital twin to track expected recovery trajectories. Deviations from this curve may indicate delayed progress or complications.

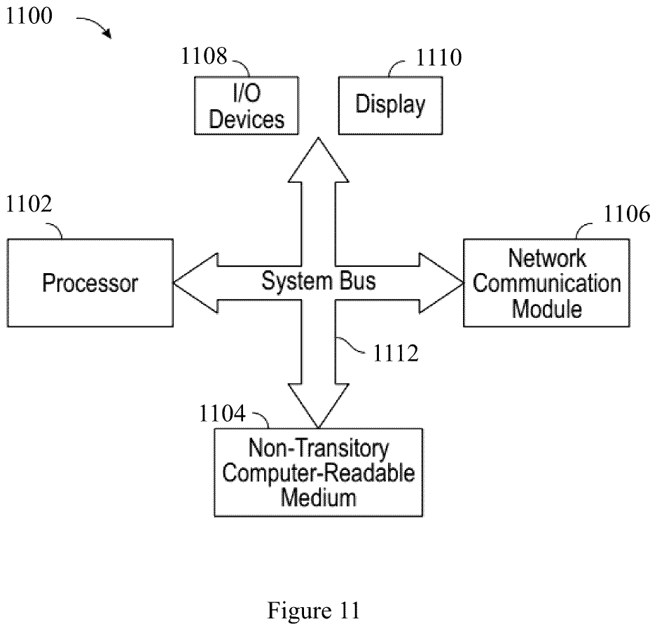

FIG. 11 shows a block diagram of a computing system 1100 in accordance with one or more embodiments. The computing system 1100 includes at least one processor 1102 (e.g., central processing unit (CPU), graphics processing unit (GPU), digital signal processor (DPS), neural proceeding unit (NPU), AI accelerator, and other processors), a non-transitory computer readable medium 1104, an optional network communication module 1106, optional input/output devices 1108, a data storage drive or device, and an optional display 1110 all interconnected via a system bus 1112. In at least one embodiment, the input/output device 1108 and the display 1110 may be combined into a single device, such as a touch-screen display. Software instructions executable by the processor 1102 for implementing software instructions stored within the computing system 1100 in accordance with the illustrative embodiments described herein, may be stored in the non-transitory computer readable medium 1104 or some other non-transitory computer-readable medium.

Although not explicitly shown in FIG. 11, it should be recognized that the computing system 1100 may be connected to one or more public and/or private networks via appropriate network connections. It will also be recognized that software instructions may also be loaded into the non-transitory computer readable medium 1104 from an appropriate storage media or via wired or wireless means. Moreover, the internal and external communication of the computing system 1100 may be accomplished through wired and/or wireless communications, including known communication protocols, Wi-Fi, 802.11 (x), Bluetooth, to name just a few.

The above description refers to a block diagram of the accompanying drawings. Alternative implementations of the example represented by the block diagram include one or more additional or alternative elements, processes and/or devices. Additionally, or alternatively, one or more of the examples blocks of the diagram may be combined, divided, re-arranged or omitted. Components represented by the blocks of the diagram are implemented by hardware, software, firmware, and/or any combination of hardware, software and/or firmware. In some examples, at least one of the components represented by the blocks is implemented by a logic circuit. As used herein, the term “logic circuit” is expressly defined as a physical device including at least one hardware component configured (e.g., via operation in accordance with a predetermined configuration and/or via execution of stored machine-readable instructions) to control one or more machines and/or perform operations of one or more machines. Examples of a logic circuit include one or more processors, one or more coprocessors, one or more microprocessors, one or more controllers, one or more digital signal processors (DSPs), one or more application specific integrated circuits (ASICs), one or more field programmable gate arrays (FPGAs), one or more microcontroller units (MCUs), one or more hardware accelerators, one or more special-purpose computer chips, and one or more system-on-a-chip (SoC) devices. Some example logic circuits, such as ASICs or FPGAs, are specifically configured hardware for performing operations (e.g., one or more of the operations described herein and represented by the flowcharts of this disclosure, if such are present). Some example logic circuits are hardware that executes machine-readable instructions to perform operations (e.g., one or more of the operations described herein and represented by the flowcharts of this disclosure, if such are present). Some example logic circuits include a combination of specifically configured hardware and hardware that executes machine-readable instructions. The above description refers to various operations described herein and flowcharts that may be appended hereto to illustrate the flow of those operations. Any such flowcharts are representative of example methods disclosed herein. In some examples, the methods represented by the flowcharts implement the apparatus represented by the block diagrams. Alternative implementations of example methods disclosed herein may include additional or alternative operations. Further, operations of alternative implementations of the methods disclosed herein may be combined, divided, re-arranged or omitted. In some examples, the operations described herein are implemented by machine-readable instructions (e.g., software and/or firmware) stored on a medium (e.g., a tangible machine-readable medium) for execution by one or more logic circuits (e.g., processor(s)). In some examples, the operations described herein are implemented by one or more configurations of one or more specifically designed logic circuits (e.g., ASIC(s)). In some examples the operations described herein are implemented by a combination of specifically designed logic circuit(s) and machine-readable instructions stored on a medium (e.g., a tangible machine-readable medium) for execution by logic circuit(s).

As used herein, each of the terms “tangible machine-readable medium,” “non-transitory machine-readable medium” and “machine-readable storage device” is expressly defined as a storage medium (e.g., a platter of a hard disk drive, a digital versatile disc, a compact disc, flash memory, read-only memory, random-access memory, etc.) on which machine-readable instructions (e.g., program code in the form of, for example, software and/or firmware) are stored for any suitable duration of time (e.g., permanently, for an extended period of time (e.g., while a program associated with the machine-readable instructions is executing), and/or a short period of time (e.g., while the machine-readable instructions are cached and/or during a buffering process)). Further, as used herein, each of the terms “tangible machine-readable medium,” “non-transitory machine-readable medium” and “machine-readable storage device” is expressly defined to exclude propagating signals. That is, as used in any claim of this patent, none of the terms “tangible machine-readable medium,” “non-transitory machine-readable medium,” and “machine-readable storage device” can be read to be implemented by a propagating signal.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

1. A system, comprising:
a plurality of sensors monitoring a person under monitoring (PUM) and an environment in which the PUM is located;
a machine learning (ML) monitoring system to receive data from the plurality of sensors and identify, using the data received from the plurality of sensors, a movement type and a set of values of movement parameters corresponding to a movement of the PUM, and to produce a movement representation representing the movement type and the set of values of movement parameters corresponding to the movement; and
a ML analysis system configured to receive the movement representations from the ML monitoring system, based at least in part on the movement representations, to identify and report changes in a condition of the PUM.

2. The system of claim 1, further comprising:
a data repository storing historical movement data for the PUM, the ML analysis system in communication with the data repository,
wherein the ML analysis system is further configured to identify changes in the condition of the PUM based on a comparison of the movement representations with the historical movement data for the PUM.

3. The system of claim 1, further comprising:
a data repository storing movement representations for a plurality of individuals, wherein the ML analysis system is further configured to identify changes in the condition of the PUM based on correlating the movement of the PUM to the movement representations for the plurality of individuals.

4. The system of claim 1, wherein the movement type is selected from the group consisting of at least one of: (h) reaching for an object, (i) picking up an object, (j) picking up an object from the floor, and (n) contacting an object.

5. The system of claim 1, wherein the movement parameters include one or more parameters selected from the group consisting of movement duration, movement distance, movement starting location, movement ending location, PUM body position prior to movement, PUM body position after movement, movement speed, movement vector, movement force, joint angles, angular velocity, acceleration, force vector, and energy expenditure.

6. The system of claim 5, wherein the movements representations include information regarding a joint-angle range or max achievable angle for a particular joint of the PUM.

7. (canceled)

8. The system of claim 1, further comprising:
a controller configured to reconfigure one or more of the plurality of sensors based on the identified movement type, wherein reconfiguring one or more of the plurality of sensors includes an action selected from the group consisting of activating a sensor, deactivating a sensor, changing a sampling rate of a sensor, changing a granularity of data collected by a sensor, and changing a focus of a sensor.

9. (canceled)

10. (canceled)

11. The system of claim 8, wherein the ML analysis system is further configured to output a risk metric based on the condition of the PUM, wherein the risk metric comprises a signal including at least one of a potential fall of the PUM, a deterioration in mobility in the PUM, an increased risk condition in the PUM, a change from a baseline behavior of the PUM, or an emergency that warrants human and/or automated response.

12. (canceled)

13. A method, comprising:
receiving data from a plurality of sensors monitoring a person under monitoring (PUM) and an environment in which the PUM is located;
determining from the data a movement type and a set of values of movement parameters corresponding to a movement of the PUM;
storing a movement representation representing the movement type and the set of values of movement parameters corresponding to the movement;
determining from the movement representations changes in a condition of the PUM.

14. The method of claim 13, wherein the movement type and the set of movement-parameter values are determined using a movement analysis machine learning system.

15. The method of claim 14, further comprising:
training the movement analysis machine learning system using a Musculo-skeletal computational model of a human body.

16. The method of claim 15, further comprising:
producing a customized musculoskeletal computational model to the PUM based applying the movements representations to the Musculo-skeletal computational model.

17. (canceled)

18. The method of claim 13, further comprising:
receiving the data from the plurality of sensors for a time period; and
determining a segment of the time period which corresponds to the movement of the PUM prior to determining the movement type and the set of movement-parameter values.

19. (canceled)

20. (canceled)

21. The method of claim 13, further comprising:
outputting a risk metric based on the condition of the PUM, wherein the risk metric comprises a signal selected from the group consisting of a potential fall of the PUM, a deterioration in mobility in the PUM, an increased risk condition in the PUM, a change from a baseline behavior of the in the PUM, and an emergency that warrants human and/or automated response.

22. The method of claim 13, further comprising:
computing a deviation metric between the movement representations and a baseline movement profile of the PUM; and
triggering an alert when the deviation metric exceeds a predetermined threshold.

23. The method of claim 13, further comprising:
encoding the identified movement type and the set of values of movement parameters in one or more machine-interpretable tokens or vectors as part of the movement representation.

24.-56. (canceled)

57. A method, comprising:
receiving, from sensors of a sensor-enabled environment (SEE), multimodal measurements characterizing movements of a person under monitoring (PUM);
generating, by a first-stage model, tokenized movement representations from the multimodal measurements;
filtering, by a personalized physics engine (PPE), movement artifacts and movements failing physics-based constraints of the PUM to produce artifact-reduced movement data;
updating, by a digital twin of the PUM, a baseline behavior model using the artifact-reduced movement data;
determining, by comparison of a current behavior to the baseline behavior model, a deviation metric; and
triggering an alert when the deviation metric exceeds a predetermined threshold.

58. The method of claim 57, further comprising:
synchronizing measurements from a plurality of sensors using time-stamps and resampling to a common window prior to generating the tokenized movement representations.

59. The method of claim 57, wherein filtering by the PPE further comprises:
producing the artifact-reduced movement data by at least one action selected from the group consisting of (a) applying joint-angle constraints, (b) analyzing contact-surface models, and (d) performing an artifact detection procedure.

60. (canceled)

61. (canceled)

62. The method of claim 57, further comprising adjusting the predetermined threshold based on at least one factor selected from the group consisting of (a) energy expenditure of the PUM, (b) measured fatigue of the PUM, (c) historical circadian behavior of the PUM, and (d) movement success metrics of the PUM.

63. (canceled)
